(19) United States
(12) Patent Application Publication
(10) Pub. No.: US 2025/0134917 A1
(43) Pub. Date: May 1, 2025

(54) COMPOSITIONS AND METHODS OF REGENERATING HEART TISSUES WITH AMINOGLYCOSIDES

(71) Applicants: The Board of Regents of The University of Texas System, Austin, TX (US); The UAB Research Foundation, Birmingham, AL (US)

(72) Inventors: Hesham A. Sadek, Dallas, TX (US); Ngoc Uyen Nhi Nguyen, Dallas, TX (US); Mahmoud Salama Ahmed, Dallas, TX (US); Diana Tomchick, Dallas, TX (US); Jianyi Zhang, Birmingham, AL (US)

(21) Appl. No.: 18/730,565

(22) PCT Filed: Jan. 20, 2023
(86) PCT No.: PCT/US2023/011241

§ 371 Date: Jul. 19, 2024

Related U.S. Application Data
(60) Provisional application No. 63/301,966, filed on Jan. 21, 2022.

(51) Int. Cl. A61K 31/7036; A61P 9/00

(52) U.S. Cl. CPC A61K 31/7036; A61P 9/00

(57) ABSTRACT

Disclosures herein are directed to methods of regenerating cardiomyocytes in heart tissue damaged as a result heart failure in a subject by administering one or more aminoglycosides to a subject in need thereof. Accordingly, disclosures herein are also toward methods of administering one or more aminoglycosides to treat heart failure in a subject in need thereof.

### CROSS REFERENCE TO RELATED APPLICATIONS

This application claims the benefit of U.S. Provisional Application No. 63/301,966, filed Jan. 21, 2022, the contents of which is herein incorporated by reference in its entirety.

### BACKGROUND

### 1. Field

The present inventive concept is directed to methods of treating heart failure in a subject by administration of with one or more aminoglycosides.

### 2. Discussion of Related Art

Heart disease encompasses heart failure, heart attack, and associated comorbidities. Heart failure (HF) diseases in particular are a major cause of morbidity and mortality worldwide. An underlying cause of systolic heart failure is the inability of the adult human myocardium to regenerate itself following injury. Although the mammalian heart is not a “terminally differentiated” organ, the low cardiomyocyte turnover rate is insufficient to restore normal cardiac function following the substantial cardiomyocyte loss that can occur with injury to the heart issue. To date, the only treatment regimens for heart failure are to prevent further damage by controlling blood pressure, reducing the occurrence of arrhythmias, and preventing blood clots from forming. There are no medications that regenerate lost or damaged myocardium. For those with severe heart tissue damage, the only treatments are invasive such as surgical repair of the left ventricle or implantation of biventricular pacemakers and cardioverter defibrillators. For subjects with advanced heart failure, such surgical interventions can be risky, and it is unclear whether such surgical treatment improves long-term outcomes. As such, there is a need for less invasive therapies that not only treat heart failure but can also improve cardiac muscle function and regenerate heart tissue.

### SUMMARY OF THE INVENTION

The present disclosure is based, in part, on the finding that pharmacological inhibition of Meis1 and HOXB13 transcriptional activity promotes cardiomyocytes regeneration after injury to heart tissue. Accordingly, provided herein are pharmaceutical compositions and methods of administering pharmaceutical compositions disclosed herein to treat heart failure in a subject.

In certain embodiments, the present disclosure provides methods for regeneration of cardiomyocytes in a heart tissue. In some embodiments, methods disclosed herein of inducing regeneration of cardiomyocytes in a heart tissue may comprise contacting a heart tissue with one or more aminoglycosides. In accordance with these embodiments, one or more aminoglycosides suitable for use in the methods of inducing regeneration of cardiomyocytes as disclosed herein may comprise amikacin, gentamicin, tobramycin, streptomycin, neomycin, kanamycin, netilmicin, paromomycin, spectinomycin, or any combination thereof. In some embodiments, methods disclosed herein may comprise contacting the heart tissue with two aminoglycosides. In some embodiments, methods disclosed herein may comprise contacting the heart tissue with paromomycin and neomycin.

In some embodiments, methods disclosed herein may comprise contacting a heart tissue with one or more aminoglycosides wherein the methods may increase cardiomyocyte proliferation. In accordance with these embodiments, contacting a heart tissue with one or more aminoglycosides may increase cardiomyocyte proliferation by about 1-fold to about 10-fold compared to a heart tissue not contacted with the one or more aminoglycosides.

In certain embodiments, the present disclosure provides methods of improving cardiac repair of heart tissue in a subject in need thereof. In some embodiments, methods of improving cardiac repair of heart tissue in a subject in need thereof may comprise administering to the subject an effective amount of one or more aminoglycosides. In some embodiments, methods of improving cardiac repair of heart tissue in a subject in need thereof may comprise administering to the subject an effective amount of one or more aminoglycosides wherein the subject has or is suspected of having a myocardial injury. In accordance with these embodiments, the one or more aminoglycosides suitable for use in the methods of improving cardiac repair of heart tissue in a subject as disclosed herein may comprise amikacin, gentamicin, tobramycin, streptomycin, neomycin, kanamycin, netilmicin, paromomycin, spectinomycin, or any combination thereof. In some embodiments, methods of improving cardiac repair of heart tissue in a subject in need thereof may comprise administering to the subject an effective amount of two aminoglycosides. In some embodiments, methods of improving cardiac repair of heart tissue in a subject in need thereof may comprise administering to the subject an effective amount of paromomycin and neomycin.

In some embodiments, methods disclosed herein may comprise administering to a subject in need thereof one or more aminoglycosides formulated in a pharmaceutical composition. In accordance with these embodiments, the one or more aminoglycosides formulated in a pharmaceutical composition as disclosed herein may further comprise at least one pharmaceutically acceptable carrier. In some embodiments, methods disclosed herein may comprise administering one or more aminoglycosides to a subject by a parenteral route.

In some embodiments, methods disclosed herein may comprise administering to a subject in need thereof one or more aminoglycosides wherein the subject has or is suspected of having a myocardial injury selected from the group consisting of arterial disease, atheroma, atherosclerosis, arteriosclerosis, coronary artery disease, arrhythmia, angina pectoris, congestive heart disease, ischemic cardiomyopathy, myocardial infarction, stroke, transient ischemic attack, aortic aneurysm, cardiopericarditis, bacterial infection, viral infection, inflammation, valvular insufficiency, and vascular clotting defects.

In some embodiments, methods of administering of an effective amount of one or more aminoglycosides to a subject as disclosed herein may increase life expectancy of the subject compared to an untreated subject with identical disease condition and predicted outcome. In some embodiments, methods of administering of an effective amount of one or more aminoglycosides to a subject as disclosed herein may improve heart function of the subject compared to an untreated subject with identical disease condition and predicted outcome. In some embodiments, methods of administering of an effective amount of one or more aminoglycosides to a subject as disclosed herein may reduce cardiac fibrosis in the subject compared to an untreated subject with identical disease condition and predicted outcome. In some embodiments, methods of administering of an effective amount of one or more aminoglycosides to a subject as disclosed herein may reverses cardiac hypertrophy in the subject compared to an untreated subject with identical disease condition and predicted outcome.

In some embodiments, methods of administering of an effective amount of one or more aminoglycosides as disclosed herein may comprise administering to a subject that is a human patient having or suspected of having a myocardial injury. In accordance with these embodiments, one or more aminoglycosides may be administered according to the methods disclosed herein to a human subject before myocardial injury, during myocardial injury, or after myocardial injury. In some embodiments, one or more aminoglycosides may be administered according to the methods disclosed herein to a human subject immediately after myocardial injury. In some embodiments, one or more aminoglycosides may be administered according to the methods disclosed herein to a human subject up to about one week after myocardial injury.

In some embodiments, one or more aminoglycosides may be administered according to the methods disclosed herein to a subject by a schedule ranging from about three times per day to about every other week. In some embodiments, one or more aminoglycosides may be administered according to the methods disclosed herein to a subject at a dose ranging from about 50 mg/kg/day to about 500 mg/kg/day.

In certain embodiments, the present disclosure provides for methods of treating heart failure in a subject in need thereof. In some embodiments, methods of treating heart failure in a subject in need thereof may comprise administering a pharmaceutical composition to the subject, wherein the pharmaceutical composition may comprise an effective amount of one or more aminoglycosides and at least one pharmaceutically acceptable carrier. In accordance with these embodiments, one or more aminoglycosides suitable for use in the methods of treating heart failure as disclosed herein may comprise amikacin, gentamicin, tobramycin, streptomycin, neomycin, kanamycin, netilmicin, paromomycin, spectinomycin, or any combination thereof. In some embodiments, methods of treating heart failure in a subject in need thereof may comprise administering two aminoglycosides. In some embodiments, methods of treating heart failure in a subject in need thereof may comprise administering paromomycin and neomycin.

In some embodiments, methods of treating heart failure in a subject in need thereof may comprise administering a pharmaceutical composition disclosed herein to the subject by a parenteral route. In some embodiments, methods of treating heart failure in a subject in need thereof may comprise administering a pharmaceutical composition disclosed herein to the subject wherein the pharmaceutical composition may comprise about 5 mg to about 500 mg of the one or more aminoglycosides. In some embodiments, methods of treating heart failure in a subject in need thereof may comprise administering a pharmaceutical composition disclosed herein to the subject wherein the pharmaceutical composition may be administered to the subject by a schedule ranging from about three times per day to about every other week.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein. Embodiments of the present inventive concept are illustrated by way of example in which like reference numerals indicate similar elements and in which:

FIGS. 1A-1L depict an exemplary process for identification of paromomycin and neomycin as FDA-approved drugs targeting MEIS1/HOXB13. FIG. 1A shows structural insights for potential druggable sites for Meis1-HOXB13. S1: HOXB13 DNA binding domain, S2: Meis1 DNA binding domain, and S3: Interacting interface between Meis1 and HOXB13 (PDB ID: 5EGO). FIG. 1B shows a schematic of an in silico screening platform for energy minimized FDA library. FIG. 1C shows interacting energies (AG) for the top 10 clinical candidates targeting S1, S2, and S3. FIG. 1D shows representative images depicting immunostaining for PH3 (green) and cardiac troponin T (red), showing the percentage of mitotic NRVMs (arrowheads). FIG. 1E shows the ratio of pH3+ myocytes to cardiac troponin T+ myocytes determined from immunostaining of NRVMs as performed in FIG. 1C. FIG. 1F shows Meis1 relative luciferase transcriptional activity in NRVMs after treatment with 10 nM, 50 nM, 100 nM, or 500 nM of neomycin, paromomycin, or PBS (Control). FIG. 1G shows HOXB13 relative luciferase transcriptional activity in NRVMs after treatment with 10 nM, 50 nM, 100 nM, or 500 nM of neomycin, paromomycin, or PBS (Control). FIG. 1H shows Meis1 relative luciferase transcriptional activity in NRVMs after treatment with 10 nM, 50 nM, 100 nM, or 500 nM of hesperidin, rutin, or DMSO (Control). FIG. 1/shows HOXB13 relative luciferase transcriptional activity in NRVMs after treatment with 10 nM, 50 nM, 100 nM, or 500 nM of hesperidin, rutin, or PBS (Control). FIG. 1J shows the chemical structure for paromomycin. FIG. 1K shows the chemical structure for Neomycin. FIG. 1L shows docked poses for neomycin (Magenta) and paromomycin (Green) against S1, S2, and S3 sites for Meis1-HOXB13 crystal structure where two different controls were used for the sake of solubility. Data are mean±s.e.m.; unpaired two-sided t-test. * P<0.05, ** P<0.01, *** P<0.001.

FIGS. 2A-2H depict an exemplary structural insights of ribostamycin (3-membered ring aminoglycoside) neomycin degradable product bound to Meis1. FIGS. 2A-2B show size exclusion chromatography and purification of Meis1 (FIG. 2A) and HOXB13 (FIG. 2B). FIGS. 2C-2D show thermal shift assays for PBS, Paromomycin, and Neomycin against Meis1 (FIG. 2C) and HOXB13 (FIG. 2D). FIG. 2E shows a surface representation for Meis1 (orange) bound to Ribostamycin (Magenta). FIG. 2F shows a ribbon cartoon representation for HOXB13 (Marine blue) and Meis1 (Orange) along with Ribostamycin (Magenta). FIG. 2G shows a close-up for Ribostamycin along with Meis1 highlighting the contributing amino acids H297.A, P298.A, and Y299.A. FIG. 2H shows a close-up for the contributing amino acid residues from HOX13 and Meis1 along with Ribostamycin. Data are mean±s.e.m.; unpaired two-sided t-test. * P<0.05, ** P<0.01, *** P<0.001.

FIGS. 3A-3M depict an exemplary increase in cardiomyocyte proliferation indices in adult mice treated with paromomycin. FIG. 3A shows a schematic for paromomycin administration to adult CD-1 mice at 200 mg/kg, i.p for 2 weeks followed by collection of hearts for histological analysis. FIG. 3B shows heart weight to body weight ratio for paromomycin-treated mice compared with controls. FIGS. 3C-3D show a representative image of anti-pH3 and anti-cardiac troponin T (anti-cTnT) co-immunostaining (FIG. 3C) and its quantitative analysis (FIG. 3D) showing a significant increase in the cardiomyocyte mitosis marker for paromomycin-treated mice, compared with controls. FIGS. 3E-3F show a representative image of AurkB and anti-cTnT co-immunostaining (FIG. 3E) and its quantitative analysis (FIG. 3F) showing a significant increase in the cardiomyocyte cytokinesis marker for paromomycin-treated mice compared with Controls. FIGS. 3G-3H show a representative image of WGA staining (FIG. 3G) and its quantitative analysis (FIG. 3H) showing a significant decrease in cardiomyocyte cell size for paromomycin-treated mice, compared with controls. FIG. 3/shows a significant increase in the cardiomyocyte cell count of paromomycin-treated mice compared with controls. FIGS. 3J-3K show a representative image of Anti-Cnx43 and nuclear staining (DAPI) co-immunostaining (FIG. 3J) and its quantitative analysis (FIG. 3K) showing a significant decrease in the bi- and multi-nucleation, while significant increase in the mono-nucleation paromomycin-treated mice, compared with controls. FIGS. 3L-3M show a representative image of single-labelled cardiomyocytes in a heat section (FIG. 3L) and its quantitative analysis (FIG. 3M) showing a significant increase in paromomycin-treated mice, compared with controls after injection of Tamoxifen. Data are mean±s.e.m.; unpaired two-sided t-test. Data were independently repeated three to six times with similar results. * P<0.05, ** P<0.01, *** P<0.001.

FIGS. 4A-4R depict an exemplary increase in cardiomyocyte proliferation indices in neonatal and adult mice treated with a combination of neomycin and paromomycin. FIG. 4A shows a schematic for paromomycin-neomycin administration to neonates CD-1 mice at 300 mg/kg, i.p from p1 till p14. Hearts were collected for histological analysis. FIGS. 4B-4C show a representative image of anti-pH3 and anti-cardiac troponin T (anti-cTnT) co-immunostaining (FIG. 4B) and its quantitative analysis (FIG. 4C) showing a significant increase in the cardiomyocyte mitosis marker for paromomycin-treated neonates compared with controls. FIGS. 4D-4E show a representative image of AurkB and anti-cTnT co-immunostaining (FIG. 4D) and its quantitative analysis (FIG. 4E) showing a significant increase in the cardiomyocyte cytokinesis marker for paromomycin-treated neonates, compared with controls. FIG. 4F shows a schematic for paromomycin-neomycin administration to adult CD-1 mice at 300 mg/kg, i.p for 2 weeks. Hearts were collected for histological analysis. FIG. 4G shows heart weight to body weight ratio shows for paromomycin-neomycin treated mice compared with controls. FIGS. 4H-4/show a representative image of anti-pH3 and anti-cardiac troponin T (anti-cTnT) co-immunostaining (FIG. 4H) and its quantitative analysis (FIG. 4I) showing a significant increase in the cardiomyocyte mitosis marker for paromomycin-neomycin treated mice compared with Controls. FIGS. 4J-4K show a representative image of AurkB and anti-cTnT co-immunostaining (FIG. 4J) and its quantitative analysis (FIG. 4K) showing a significant increase in the cardiomyocyte cytokinesis marker for paromomycin-neomycin treated mice compared with Controls. FIGS. 4L-4M show a representative image of WGA staining (FIG. 4L) and its quantitative analysis (FIG. 4M) showing a significant decrease in cardiomyocyte cell size for paromomycin-neomycin treated mice, compared with Controls. FIG. 4N shows a significant increase in the cardiomyocyte cell count of paromomycin-neomycin treated mice compared with controls. FIGS. 4O-4P show a representative image of Anti-Cnx43 and nuclear staining (DAPI) co-immunostaining (FIG. 4O) and its quantitative analysis (FIG. 4P) showing a significant decrease in the bi- and multi-nucleation, while significant increase in the mono-nucleation paromomycin-neomycin treated mice, compared with controls. FIGS. 4Q-4R show a representative image of single-labelled cardiomyocytes in a heat section (FIG. 4Q) and its quantitative analysis (FIG. 4R) showing a significant increase in paromomycin-neomycin treated mice, compared with controls after injection of 4-Hydroxy Tamoxifen. Data are mean±s.e.m.; unpaired two-sided t-test. Data were independently repeated three to six times with similar results. * P<0.05, ** P<0.01, *** P<0.001.

FIGS. 5A-5G depict an exemplary prevention of remodeling in MI mouse model treated with aminoglycosides. FIG. 5A shows a schematic of a MI mouse model, where one week after MI was induced by left anterior descending ligation, mice were injected with PBS (Control), Paromomycin (200 mg/kg, i.p.), and Paromomycin-Neomycin (300 mg/kg, i.p., the dose was divided to be administered twice daily) for 8 weeks. The cardiac function was assessed weekly by echocardiography. Hearts were collected after 8 weeks post-MI for histological analysis. FIGS. 5B-5C show representative echocardiography images for control, paromomycin, and paromomycin-neomycin treated hearts at 1 week (Pre-injection; FIG. 5B) and 8 weeks post-MI (FIG. 5C). FIG. 5D shows a significant decrease in the wet to dry lung ratio of paromomycin and paromomycin-neomycin treated mice compared to controls. FIG. 5E shows an echocardiography analysis of left ventricular ejection fraction (LVEF) depicting maintained LVEF post-MI in paromomycin-treated mice compared to controls. However, higher LVEF post-MI in paromomycin-neomycin treated mice was recorded, compared to controls. FIG. 5F shows Trichrome staining of hearts, 8-weeks post-MI, depicting a marked decrease in LV dilatation and remodelling of paromomycin-neomycin treated hearts, compared with control MCM hearts. FIG. 5G shows a non-significant decline in the infarcted size of paromomycin-treated hearts while paromomycin-neomycin treated hearts showed a significant decrease in the infarcted size midline length, compared to controls. Data are mean±s.e.m.; unpaired two-sided t-test. * P<0.05, ** P<0.01, *** P<0.001.

FIGS. 6A-6J depict an exemplary prevention of remodeling in MI pig model treated with aminoglycosides. FIG. 6A shows a schematic of a MI pig model, where one week after MI was induced, pigs were administered paromomycin-neomycin (15-16 mg/kg, i.v.) and every 2 weeks thereafter until 4 weeks post-surgery. FIG. 6B shows a graph depicting fraction shortening normalized to baseline in Paro/Neo combo-treated pigs. FIGS. 6C-6E show graphs of heart weight to body weight ratio (FIG. 6C), heart weight (FIG. 6D), and body weight (FIG. 6E) for paromomycin-neomycin treated pigs compared with controls. FIG. 6F shows a representative image of serial sections of whole pig heart collected from paromomycin-neomycin treated pigs compared with controls. FIG. 6G shows the ratio of scar size to left ventricle size in whole pig heart collected from paromomycin-neomycin treated pigs compared with controls. FIG. 6H shows a graph depicting fraction shortening normalized to baseline in Paro/Neo combo-treated pigs. FIG. 6I shows a representative image of anti-Ki67 and anti-cardiac troponin T (anti-cTnT) co-immunostaining in pig heart. FIG. 6J shows a representative image of anti-pH3 and anti-cardiac troponin T (anti-cTnT) co-immunostaining in pig heart.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

Heart failure (HF) is a clinical syndrome caused by structural and functional defects in myocardium resulting in impairment of ventricular filling or the ejection of blood. The most common cause for HF is reduced left ventricular myocardial function as characterized by decreased LVEF; however, dysfunction of the pericardium, myocardium, endocardium, heart valves or great vessels alone or in combination is also associated with HF. One of the major pathogenic mechanisms leading to HF includes the inability of the adult heart to regenerate lost or damaged myocardium following an injury to the heart tissue. Accordingly, treatment regimens for regeneration of heart tissue, particularly pharmacological treatments which are less invasive, are needed as incidences of heart disease are steadily increasing worldwide.

The present disclosure is based, in part, on the finding that pharmacological inhibition of Meis1 and HOXB13 transcriptional activity by administration of one or more aminoglycosides promotes cardiomyocytes regeneration after injury to heart tissue. Accordingly, provided herein are pharmaceutical compositions comprising aminoglycosides and methods of administering pharmaceutical compositions comprising aminoglycosides herein to treat heart failure in a subject.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

As used herein, “an effective amount” refers to the amount of each active agent required to confer therapeutic effect on the subject, either alone or in combination with one or more other active agents.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (See, e.g., Batzer et al., Nucleic Acid Res. 19:5081 (1991)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

It should also be understood that, unless clearly indicated to the contrary, in any methods claimed herein that include more than one step or act, the order of the steps or acts of the method is not necessarily limited to the order in which the steps or acts of the method are recited.

### II. Aminoglycosides

The present disclosure provides compositions comprising and methods of use of on or more aminoglycosides. Aminoglycosides are potent, broad-spectrum antibiotics that act through inhibition of protein synthesis. Aminoglycoside compounds, as described herein, may encompass any compound classified as an aminoglycoside, pharmaceutically acceptable salts or esters thereof, analogues thereof, including prodrugs thereof, and the like. In some embodiments herein, aminoglycosides may be natural compounds, semisynthetic compounds, synthetic compounds, or any combination thereof. In some embodiments, aminoglycoside compounds for use herein may be streptomycin, neomycin, kanamycin, gentamicin, netilmicin, tobramycin, amikacin, arbekacin, plazomicin, paromomycin, spectinomycin, or any combination thereof.

In some embodiments, aminoglycoside compounds described herein, where applicable, can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981). The disclosure additionally encompasses aminoglycoside compounds described herein as individual isomers substantially free of other isomers, and alternatively, as mixtures of various isomers. In some embodiments, aminoglycoside compounds used in the methods disclosed herein may be (R)-isomers. Alternatively, aminoglycoside compounds used in the methods disclosed herein may be(S)-isomers. In some embodiments, aminoglycoside compounds used herein may be a mixture of (R) and(S) isomers.

In some embodiments, aminoglycosides used in the compositions and methods disclosed herein can be in the form of an ester prodrug. The term “ester” herein can refer a compound which is produced by modifying a functional group (e.g. hydroxyl, carboxyl, amino or the like group). Examples of the “ester” include “esters formed with a hydroxyl group” and “esters formed with a carboxyl group.” The term “ester” can mean an ester whose ester residue is a “conventional protecting group” or a “protecting group removable in vivo by a biological method such as hydrolysis”. In some embodiments, the term “conventional protecting group” can mean a protecting group removable by a chemical method such as hydrogenolysis, hydrolysis, electrolysis or photolysis. In some embodiments, the term “protecting group removable in vivo by a biological method such as hydrolysis” can mean a protecting group removable in vivo by a biological method such as hydrolysis to produce a free acid or its salt.

In some embodiments, aminoglycosides used in the compositions and methods disclosed can be in the form of a pharmaceutically acceptable salt. By “salt” or “pharmaceutically acceptable salt”, it is meant those salts which are, within the scope of sound medical judgment, suitable for use in contact with the tissues of humans and lower animals without undue toxicity, irritation, and allergic response, commensurate with a reasonable benefit to risk ratio, and effective for their intended use. A “pharmacologically acceptable salt” can refer to a salt, which can be formed when an aminoglycoside compound has an acidic group such as carboxyl or a basic group such as amino or imino. In some embodiments, an aminoglycoside salt formed with an acidic group herein can include alkali metal salts such as a sodium salt, potassium salt or lithium salt, alkaline earth metal salts such as a calcium salt or magnesium salt, metal salts such as an aluminum salt or iron salt; amine salts, e.g., inorganic salts such as an ammonium salt and organic salts such as a t-octylamine salt, dibenzylamine salt, morpholine salt, glucosamine salt, phenylglycine alkyl ester salt, ethylenediamine salt, N-methylglucamine salt, guanidine salt, diethylamine salt, triethylamine salt, dicyclohexylamine salt, N,N′-dibenzylethylenediamine salt, chloroprocaine salt, procaine salt, diethanolamine salt, N-benzylphenethylamine salt, piperazine salt, tetramethylammonium salt or tris(hydroxymethyl)aminomethane salt; and amino acid salts such as a glycine salt, lysine salt, arginine salt, ornithine salt, glutamate or aspartate. In some embodiments, a aminoglycoside salt formed with a basic group herein can include hydro-halides such as a hydrofluoride, hydrochloride, hydrobromide or hydroiodide, inorganic acid salts such as a nitrate, perchlorate, sulfate or phosphate; lower alkanesulfonates such as a methanesulfonate, trifluoromethanesulfonate or ethanesulfonate, arylsulfonates such as a benzenesulfonate or p-toluenesulfonate, organic acid salts such as an acetate, malate, fumarate, succinate, citrate, ascorbate, tartrate, oxalate or maleate; and amino acid salts such as a glycine salt, lysine salt, arginine salt, ornithine salt, glutamate or aspartate. In certain embodiments, when a pharmacologically acceptable salt of an aminoglycoside remains in the atmosphere or is recrystallized, it can absorb water to form a hydrate of use in formulations disclosed herein.

In some embodiments, aminoglycosides used in the compositions and methods disclosed can be in the form of another aminoglycoside derivative. In some embodiments, the term “other derivative” can mean a derivative of the aminoglycoside compound other than the above-described “ester” or the above-described “pharmacologically acceptable salt” which can be formed, if it has an amino and/or carboxyl group or other conjugate form or other active derivative thereof.

### III. Pharmaceutical Compositions

The present disclosure provides for pharmaceutical compositions comprising one or more aminoglycosides disclosed herein. In certain embodiments, pharmaceutical compositions disclosed herein may further compromise one or more pharmaceutically acceptable diluent(s), excipient(s), or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art. Pharmaceutical compositions disclosed herein may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, and combinations thereof.

In some embodiments, pharmaceutical compositions disclosed herein may be formulated in a conventional manner using one or more physiologically acceptable carriers including excipients and auxiliaries which can facilitate processing of active components into preparations which can be used pharmaceutically. In some embodiments, proper formulation of pharmaceutical compositions disclosed herein may be dependent upon the route of administration chosen. In some embodiments, any of the well-known techniques, carriers, and excipients may be used as suitable and as understood in the art. A summary of pharmaceutical compositions described herein may be found (but are not limited to), for example, in Hoover, John E., REMINGTON'S PHARMACEUTICAL SCIENCES, Mack Publishing Co., Easton, Pa. 1995; Liberman, H. A. and Lachman, L., Eds., PHARMACEUTICAL DOSAGE FORMS, Marcel Decker, New York, N.Y., 1980; and PHARMACEUTICAL DOSAGE FORMS AND DRUG DELIVERY SYSTEMS, Seventh Ed. (Lippincott Williams & Wilkins 1999), herein incorporated by reference in their entirety for such disclosure.

In certain embodiments, pharmaceutical compositions disclosed herein may be formulated in conventional manner using one or more physiologically acceptable carriers comprising excipients and auxiliaries to facilitate processing of genetically modified endothelial progenitor cells into preparations which can be used pharmaceutically. In some embodiments, any of the well-known techniques, carriers, and excipients may be used as suitable and as understood in the art.

In some embodiments, pharmaceutical compositions described herein may be an aqueous suspension comprising one or more polymers as suspending agents. In some aspects, polymers suitable for use herein may include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In other aspects, compositions disclosed herein may comprise at least 5%, at least 10%, at least 20%, at least 25%, at least 30%, at least 35%, at least 40%, at least 45%, at least 50% total amount of polymers as suspending agent(s) by total weight of the composition

In certain embodiments, pharmaceutical compositions disclosed herein may comprise a viscous formulation. In some aspects, viscosity of the pharmaceutical composition may be increased by the addition of one or more gelling or thickening agents. In other aspects, pharmaceutical compositions disclosed herein may comprise one or more gelling or thickening agents in an amount to provide a sufficiently viscous formulation to remain on treated tissue. In still other aspects, pharmaceutical compositions disclosed herein may comprise at least 5%, at least 10%, at least 20%, at least 25%, at least 30%, at least 35%, at least 40%, at least 45%, at least 50% total amount of gelling or thickening agent(s) by total weight of the composition. In yet other aspects, suitable thickening agents can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or combinations thereof. In specific embodiments, suitable thickening agent may be carboxymethylcellulose.

In certain embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, at least 50% total amount of one or more agents by total weight of the composition. In other aspects, one or more of these agents may be added to improve the performance, efficacy, safety, shelf-life and/or other property of the muscarinic antagonist composition of the invention. In some aspects, additives contemplated herein may be biocompatible, and will not be harsh, abrasive, or allergenic.

In some embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In other aspects, pharmaceutical compositions disclosed herein may comprise at least 5%, at least 10%, at least 20%, at least 25%, at least 30%, at least 35%, at least 40%, at least 45%, at least 50% total amount of one or more acidifying agents by total weight of the composition.

In some embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In other aspects, pharmaceutical compositions disclosed herein may comprise at least 5%, at least 10%, at least 20%, at least 25%, at least 30%, at least 35%, at least 40%, at least 45%, at least 50% total amount of one or more alkalizing agents by total weight of the composition.

In some embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more antioxidants by total weight of the composition.

In some embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In another aspect, pharmaceutical compositions disclosed herein may comprise at least 5%, at least 10%, at least 20%, at least 25%, at least 30%, at least 35%, at least 40%, at least 45%, at least 50% total amount of one or more buffering agents by total weight of the composition. In other aspects, the amount of one or more buffering agents may depend on the desired pH level of a composition. In some embodiments, pharmaceutical compositions disclosed herein may have a pH of about 6 to about 9. In other embodiments, pharmaceutical compositions disclosed herein may have a pH greater than about 8, greater than about 7.5, greater than about 7, greater than about 6.5, or greater than about 6. In some embodiments, pharmaceutical compositions disclosed herein may have a pH greater than about 6.8.

In some embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In other aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition.

In some embodiments, pharmaceutical compositions disclosed herein may comprise one or more surface-acting reagents or detergents. In some aspects, surface-acting reagents or detergents may be synthetic, natural, or semi-synthetic. In other aspects, compositions disclosed herein may comprise anionic detergents, cationic detergents, zwitterionic detergents, ampholytic detergents, amphoteric detergents, nonionic detergents having a steroid skeleton, or a combination thereof. In still other aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition.

In some embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition.

In some embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some embodiments, a vapor pressure depression method is used to calculate the osmolarity of the compositions disclosed herein. In some aspects, an amount of one or more tonicity agents to be added to a pharmaceutical composition disclosed herein may result in a final pharmaceutical composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In other aspects, a pharmaceutical composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In still other aspects, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition.

In some embodiments, pharmaceutical compositions disclosed herein may be formulated for parenteral administration by injection. In some aspects, parenteral administration by injection can be by bolus injection and/or continuous infusion. In some embodiments, pharmaceutical compositions disclosed herein may be formulated for parenteral administration by intracardiac injection. As used herein, the term “intracardiac injection” refers to an injection given directly into the heart muscles or ventricles. In some embodiments, pharmaceutical compositions disclosed herein may be formulated for parenteral administration by catheter-based intracoronary infusion. In some embodiments, pharmaceutical compositions disclosed herein may formulated for parenteral administration by pericardial injection.

In some embodiments, pharmaceutical compositions disclosed herein that are formulations for injection may be presented in unit dosage form. In some aspects, a unit dosage form may be in ampoules and or in multi-dose containers. In other aspects, pharmaceutical compositions disclosed herein may be suspensions, solutions or emulsions in oily or aqueous vehicles. In still other aspects, pharmaceutical compositions disclosed herein may contain formulary agents such as suspending, stabilizing and/or dispersing agents. In yet other aspects, pharmaceutical compositions disclosed herein may be presented in unit-dose or multi-dose containers. Non-limiting examples of unit-dose or multi-dose containers include sealed ampoules and vials. In some aspects, pharmaceutical compositions disclosed herein may be stored in powder form or in a freeze-dried (lyophilized) condition requiring only the addition of the sterile liquid carrier immediately prior to use. In other aspects, pharmaceutical compositions disclosed herein may be extemporaneous injection solutions and suspensions may be prepared from sterile powders, granules, tablets or a combination thereof. In still other aspects, pharmaceutical compositions disclosed herein may be cryofrozen prior to storage. As used herein, “cryofrozen” refers to and/or describes cryopreservation biological samples frozen in a manner that maintains vitality and subsequently thawed out again as needed while maintaining vitality. In some aspects, pharmaceutical compositions disclosed herein may be cryofrozen and stored for up to 1 week, up to 4 weeks, up to 8 weeks, up to 16 weeks, up to 25 weeks, up to 50 weeks, up to 100 weeks, or up to 200 weeks while maintaining vitality.

In certain embodiments, pharmaceutical compositions disclosed herein for parenteral administration can include aqueous and non-aqueous (oily) sterile injection solutions of the compositions which may contain antioxidants, buffers, bacteriostats and solutes which render the formulation isotonic with the blood of the intended recipient; and aqueous and non-aqueous sterile suspensions which may include suspending agents and thickening agents. In some aspects, pharmaceutical compositions disclosed herein may include lipophilic solvents or vehicles. Non-limiting examples of vehicles include fatty oils such as sesame oil, or synthetic fatty acid esters, such as ethyl oleate or triglycerides, or liposomes.

In some embodiments, pharmaceutical compositions disclosed herein may be aqueous injection suspensions. In some aspects, pharmaceutical compositions disclosed herein may contain substances which increase the viscosity of the suspension, such as sodium carboxymethyl cellulose, sorbitol, or dextran. In other aspects, pharmaceutical compositions disclosed herein may comprise suitable stabilizers or agents which increase the solubility of the enzymes and fining agents to allow for the preparation of highly concentrated solutions.

### IV. Methods of Use

The present disclosure provides methods of regenerating a heart tissue. In some embodiments, methods disclosed here can regenerate a heart tissue by contacting the heart tissue with one or more aminoglycosides and/or a composition comprising one or more aminoglycosides as disclosed herein. In some embodiments, methods disclosed here can regenerate cardiomyocytes within a heart tissue by contacting the cardiomyocytes with one or more aminoglycosides and/or a composition comprising one or more aminoglycosides as disclosed herein.

In some embodiments, methods disclosed here can regenerate cardiomyocytes by contacting the cardiomyocytes with a total concentration of aminoglycosides ranging from about 1 μM to about 50 μM, about 5 μM to about 45 μM, or about 10 μM to about 30 μM. In some embodiments, methods disclosed here can regenerate cardiomyocytes within a heart tissue by contacting the cardiomyocytes with a total concentration of aminoglycosides of about 1 μM, about 5 μM, about 10 μM, about 15 μM, about 20 μM, about 25 μM, about 30 μM, about 35 μM, about 40 μM, about 45 μM, or about 50 μM.

In some embodiments, methods disclosed here can regenerate cardiomyocytes by increasing the number of cardiomyocytes by about 1-fold to about 50-fold following contact with one or more aminoglycosides and/or a composition comprising one or more aminoglycosides as disclosed herein. In some embodiments, methods disclosed here can regenerate cardiomyocytes by increasing the number of cardiomyocytes by about 1-fold, about 2-fold, about 3-fold, about 4-fold, about 5-fold, about 6-fold, about 10-fold, about 12-fold, about 14-fold, about 16-fold, about 18-fold, about 20-fold, about 25-fold, about 30-fold, about 35-fold, about 40-fold, about 45-fold, or about 50-fold following contact with one or more aminoglycosides and/or a composition comprising one or more aminoglycosides as disclosed herein.

The present disclosure also provides methods of treating heart disease in a subject in need thereof. A suitable subject includes a human, a livestock animal, a companion animal, a lab animal, or a zoological animal. In one embodiment, the subject may be a rodent, e.g., a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human. In certain embodiments, a subject in need may have been diagnosed with at least one heart disease. In some aspects, the subject may have HF. In other aspects, the subject may have or is suspected of having a myocardial infarction (MI). In some aspects, the subject may have ischemic heart tissue.

In certain embodiments, compositions disclosed herein (e.g., one or more aminoglycosides) may be administered by parenteral administration. As used herein, “by parenteral administration” refers to administration of the compositions disclosed herein via a route other than through the digestive tract. In some embodiments, compositions disclosed herein may be administered by parenteral injection. In some aspects, administration of the disclosed compositions by parenteral injection may be by subcutaneous, intramuscular, intravenous, intraperitoneal, intracardiac, intraarticular, or intracavernous injection. In other aspects, administration of the disclosed compositions by parenteral injection may be by slow or bolus methods as known in the field. In some embodiments, the route of administration by parenteral injection can be determined by the target location. In some aspects, compositions disclosed herein may be formulated for parenteral administration by intracardiac injection. In some other aspects, compositions disclosed herein may be formulated for parenteral administration by catheter-based intracoronary infusion. In still some other aspects, compositions disclosed herein may formulated for parenteral administration by pericardial injection.

In some embodiments, the dose of compositions disclosed herein (e.g., one or more aminoglycosides) to be administered is not particularly limited, and may be appropriately chosen depending on conditions such as a purpose of preventive and/or therapeutic treatment, a type of a disease, the body weight or age of a subject, severity of a disease and the like. In some embodiments, administration of a dose of a composition disclosed herein may comprise an effective amount of the composition disclosed herein. As used herein, the term “effective amount” refers to an amount of administered composition that treats heart disease, reduces presentation of at least one symptom associated with heart disease, reverses/prevents cardio fibrosis, reverse/prevent dilation of at least one heart ventricle, reduces total heart weight, improved heart function, increases survivability, or a combination thereof. An effective amount of a composition disclosed herein to be delivered to a subject may be an amount that does not result in undesirable systemic side effects.

In some embodiments, a composition disclosed herein (e.g., one or more aminoglycosides) may be administered to a subject in need thereof at a total dose of aminoglycoside ranging from about 1 mg/kg/day to about 800 mg/kg/day, about 2 mg/kg/day to about 600 mg/kg/day, or about 4 mg/kg/day to about 200 mg/kg/day. In some embodiments, a composition disclosed herein (e.g., one or more aminoglycosides) may be administered to a subject in need thereof at a total dose of aminoglycoside of about 1 mg/kg/day, about 5 mg/kg/day, about 10 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 150 mg/kg/day, about 200 mg/kg/day, about 300 mg/kg/day, about 350 mg/kg/day, about 400 mg/kg/day, about 450 mg/kg/day, about 500 mg/kg/day, about 550 mg/kg/day, about 600 mg/kg/day, about 650 mg/kg/day, about 700 mg/kg/day, about 750 mg/kg/day, or about 800 mg/kg/day.

In some embodiments, a composition disclosed herein (e.g., one or more aminoglycosides) may be administered to a subject in need thereof once. In some embodiments, a composition disclosed herein may be administered to a subject in need thereof more than once. In other embodiments, a first administration of a composition disclosed herein may be followed by a second administration of a composition disclosed herein. In some embodiments, a first administration of a composition disclosed herein may be followed by a second and third administration of a composition disclosed herein. In some embodiments, a first administration of a composition disclosed herein may be followed by a second, third, and fourth administration of a composition disclosed herein. In some embodiments, a first administration of a composition disclosed herein may be followed by a second, third, fourth, and fifth administration of a composition disclosed herein.

The number of times a composition herein (e.g., one or more aminoglycosides) may be administered to a subject in need thereof can depend on the discretion of a medical professional, the severity of the heart disease, and the subject's response to the formulation. In some embodiments, a composition disclosed herein may be administered continuously; alternatively, the dose of aminoglycoside being administered may be temporarily reduced or temporarily suspended for a certain length of time (i.e., a “drug holiday”). In some aspects, the length of the drug holiday can vary between 2 days and 1 year, including by way of example only, 2 days, 1 week, 1 month, 6 months, and 1 year. In other aspects, dose reduction during a drug holiday may be from 10%-100%, including by way of example only 10%, 25%, 50%, 75%, and 100%.

In some embodiments, the desired daily dose of compositions disclosed herein (e.g., one or more aminoglycosides) may be presented in a single dose or as divided doses administered simultaneously (or over a short period of time) or at appropriate intervals. In other embodiments, administration of a composition disclosed herein may be administered to a subject about once a day, about twice a day, about three times a day. In still other embodiments, administration of a composition disclosed herein may be administered to a subject at least once a day, at least once a day for about 2 days, at least once a day for about 3 days, at least once a day for about 4 days, at least once a day for about 5 days, at least once a day for about 6 days, at least once a day for about 1 week, at least once a day for about 2 weeks, at least once a day for about 3 weeks, at least once a day for about 4 weeks, at least once a day for about 8 weeks, at least once a day for about 12 weeks, at least once a day for about 16 weeks, at least once a day for about 24 weeks, at least once a day for about 52 weeks and thereafter. In a preferred embodiment, administration of a composition disclosed herein may be administered to a subject once a day for about 4 weeks.

In some embodiments, a composition as disclosed may be initially administered followed by a subsequent administration of one for more different compositions or treatment regimens. In other embodiments, a composition as disclosed may be administered after administration of one for more different compositions or treatment regimens.

In certain embodiments, the number of viable cardiomyocytes in an injured heart tissue of a subject treated according to the methods disclosed herein may be increased compared to the number of viable cardiomyocytes in an injured heart tissue an untreated subject with identical disease condition and predicted outcome. In some embodiments, the number of viable cardiomyocytes in an injured heart tissue of a subject treated according to the methods disclosed herein may be increased by at least about 5%, at least about 10%, at least about 20%, or at least about 25% compared to the number of viable cardiomyocytes in an injured heart tissue an untreated subject with identical disease condition and predicted outcome. In some embodiments, the number of viable cardiomyocytes in an injured heart tissue of a subject treated according to the methods disclosed herein may be increased in an amount ranging from about 1% to about 100%, about 5% to about 95%, or about 10% to about 90% compared to the number of viable cardiomyocytes in an injured heart tissue an untreated subject with identical disease condition and predicted outcome. In some embodiments, the number of viable cardiomyocytes in an injured heart tissue of a subject treated according to the methods disclosed herein may be increased by about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to the number of viable cardiomyocytes in an injured heart tissue an untreated subject with identical disease condition and predicted outcome.

In some embodiments, at least one measurement of heart function may be improved in a subject treated according to the methods disclosed herein compared to the least one measurement of heart function of an untreated subject with identical disease condition and predicted outcome. In some embodiments, LVEF may be improved in a subject treated according to the methods disclosed herein compared to the LVEF of an untreated subject with identical disease condition and predicted outcome. In some embodiments, LVEF may be improved by at least about 5%, at least about 10%, at least about 20%, or at least about 25% in a subject treated according to the methods disclosed herein compared to the LVEF of an untreated subject with identical disease condition and predicted outcome. In some embodiments, LVEF may be improved by about 1% to about 100%, by about 5% to about 95%, or by about 10% to about 90% in a subject treated according to the methods disclosed herein compared to the LVEF of an untreated subject with identical disease condition and predicted outcome. In some embodiments, LVEF may be improved by about 1%, by about 5%, by about 10%, by about 15%, by about 20%, by about 25%, by about 30%, by about 35%, by about 40%, by about 45%, by about 50%, by about 55%, by about 60%, by about 65%, by about 70%, by about 75%, by about 80%, by about 85%, by about 90%, by about 95%, or by about 100% in a subject treated according to the methods disclosed herein compared to the LVEF of an untreated subject with identical disease condition and predicted outcome.

In some embodiments, life expectancy (or “survival rate”) may be improved in a subject treated according to the methods disclosed herein compared to the life expectancy of an untreated subject with identical disease condition and predicted outcome. In some embodiments, life expectancy may be improved in a subject treated according to the methods disclosed herein by at least about 5%, at least about 10%, at least about 20%, or at least about 25% compared to the life expectancy of an untreated subject with identical disease condition and predicted outcome. In some embodiments, life expectancy may be improved in a subject treated according to the methods disclosed herein by about 1%, by about 5%, by about 10%, by about 15%, by about 20%, by about 25%, by about 30%, by about 35%, by about 40%, by about 45%, by about 50%, by about 55%, by about 60%, by about 65%, by about 70%, by about 75%, by about 80%, by about 85%, by about 90%, by about 95%, or by about 100% compared to the life expectancy of an untreated subject with identical disease condition and predicted outcome.

### V. Kits

The present disclosure also provides kits for use in treating or alleviating a target disease, such as heart failure as described herein. Such kits can include one or more containers comprising one or more aminoglycosides, and/or any pharmaceutical composition of those described herein. In some embodiments, one or more aminoglycosides disclosed herein may be co-used with a second therapeutic agent.

In some embodiments, kits herein can comprise instructions for use in accordance with any of the methods described herein. The included instructions can comprise a description of administration of the compositions disclosed (e.g., one or more aminoglycosides) herein, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering a composition disclosed herein to an individual at risk of the target disease.

In some embodiments, instructions relating to the use of a composition disclosed can include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The label or package insert indicates that the composition may be used for treating heart failure.

In some embodiments, kits disclosed herein can be in suitable packaging. Suitable packaging includes, but is not limited to, vials, bottles, jars, flexible packaging (e.g., sealed Mylar or plastic bags), and the like. Also contemplated are packages for use in combination with a specific device, such as an infusion device such as a minipump. In some embodiments, a kit herein may have a sterile access port (for example the container may be an intravenous solution bag or a vial having a stopper pierceable by a hypodermic injection needle). In some embodiments, a container in a kit herein may also have a sterile access port (for example the container may be an intravenous solution bag or a vial having a stopper pierceable by a hypodermic injection needle). In some embodiments, at least one active agent in the composition is an aminoglycoside as those described herein.

In some embodiments, kits herein may optionally provide additional components such as buffers and interpretive information. In some embodiments, a kit may comprise a container and a label or package insert(s) on or associated with the container. In some embodiments, the invention provides articles of manufacture comprising contents of the kits described above.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure. Example 1.

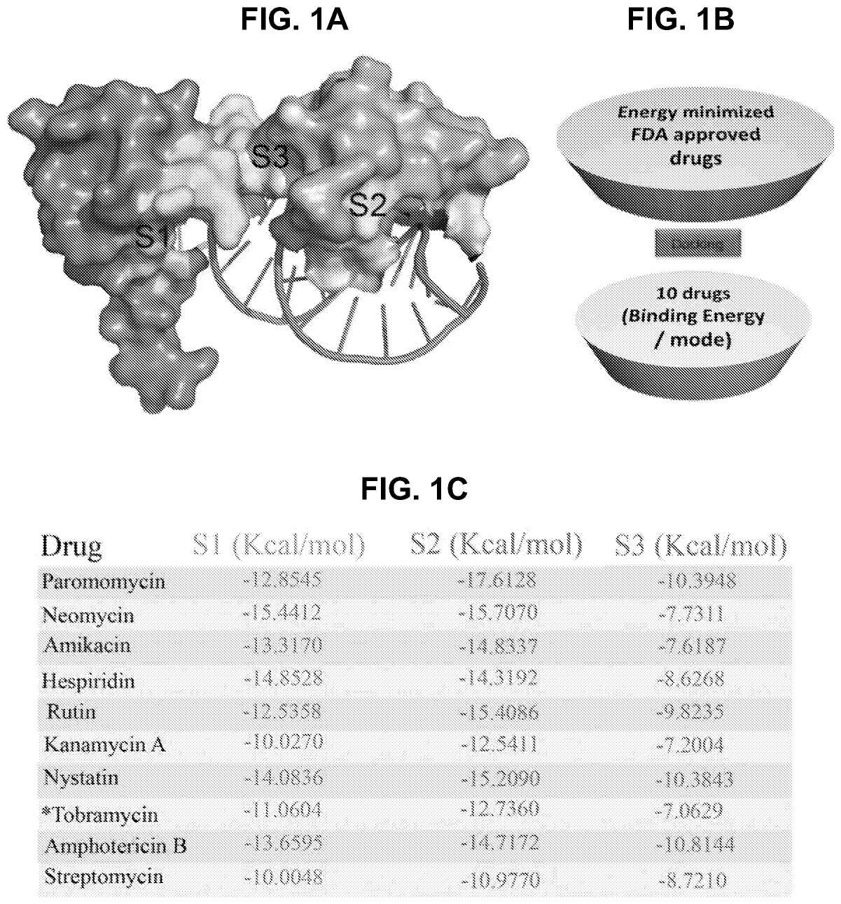

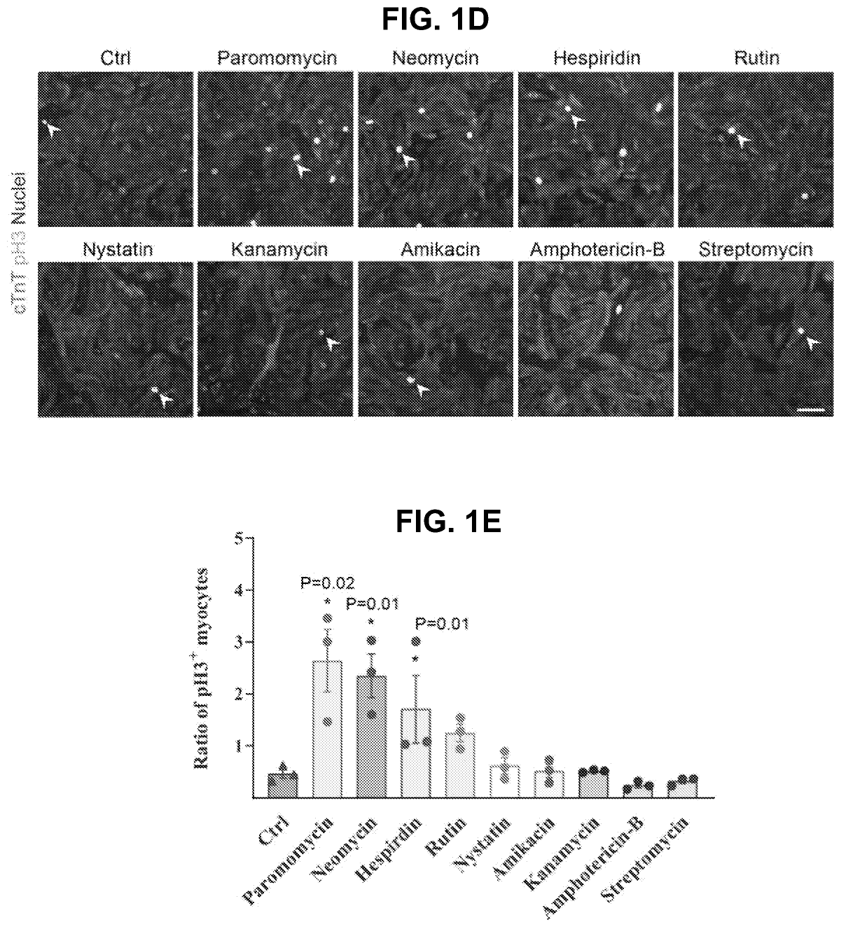

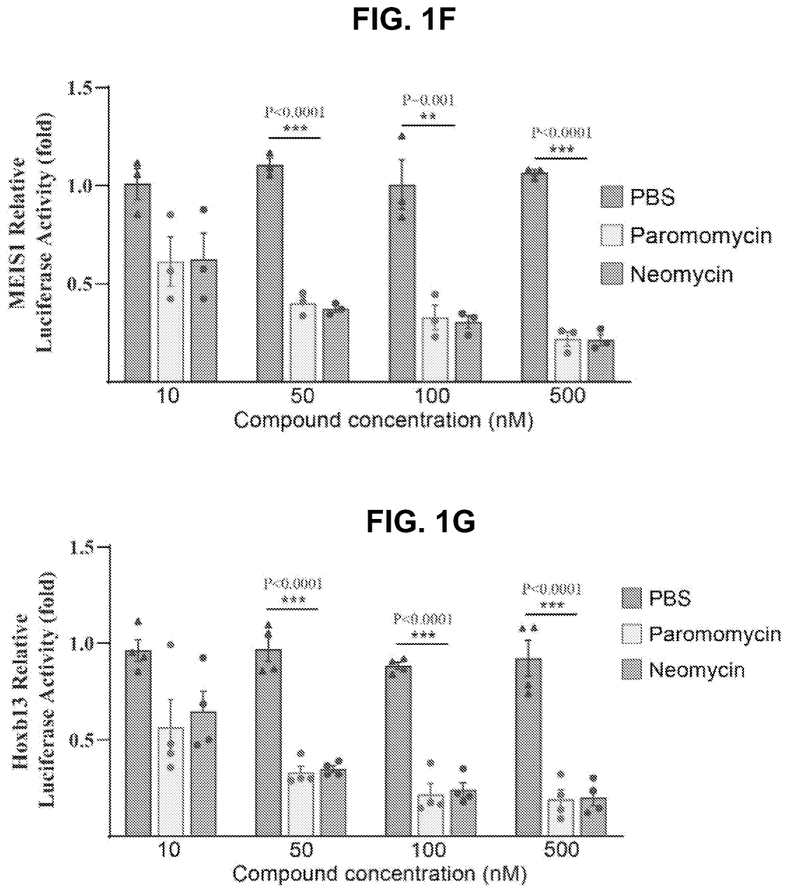

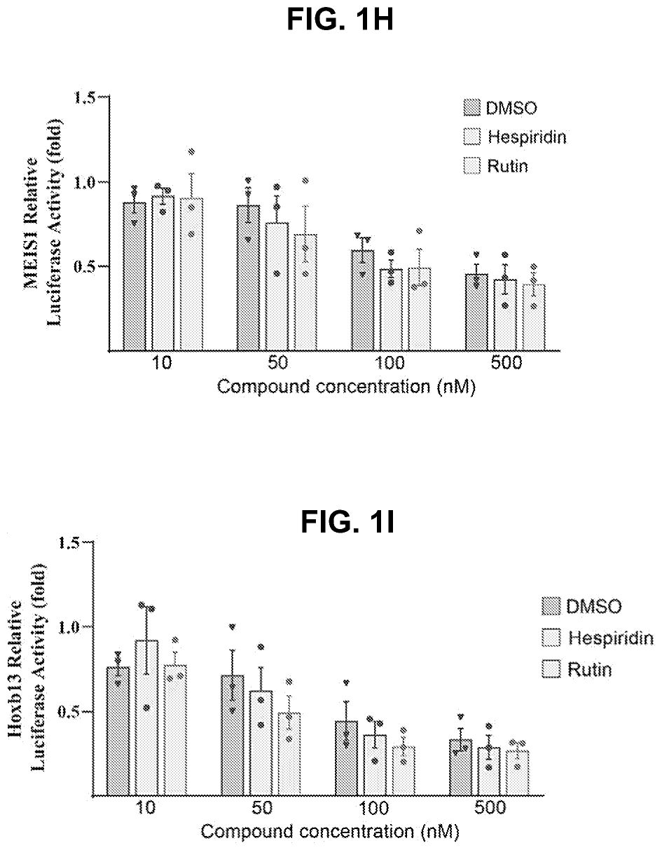

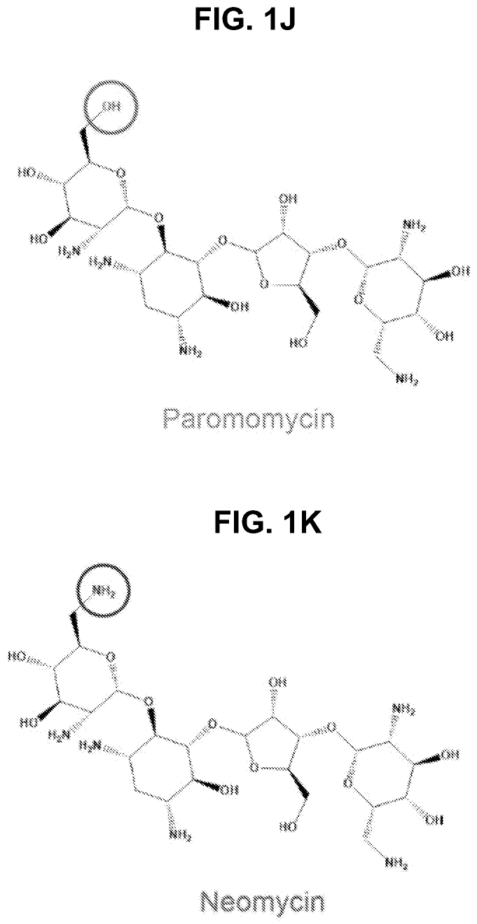

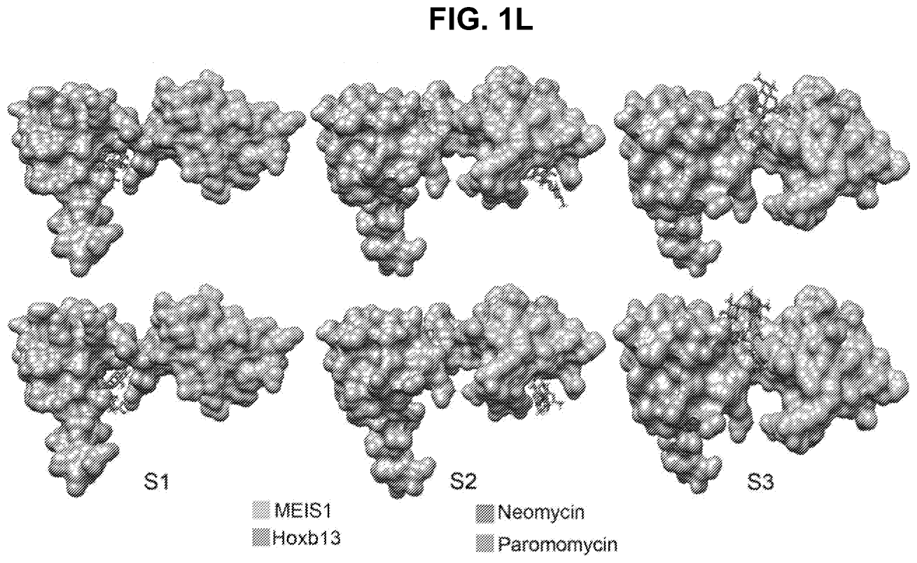

In an exemplary method, structure-based drug repurposing was used to identify small molecules targeting the Meis1-HOXB13 DNA binding domain. In brief, in silico virtual screening was performed for FDA approved drugs targeting Meis1-HOXB13 DNA binding domain using a US FDA-approved drug database was downloaded (https://go.drugbank.com/) and three-dimensional structures that were energy minimized using MMFF94 force field. Structural analysis revealed three potential targetable sites, S1 (HOXB13 bound to DNA), S2 (Meis1 bound to DNA), and S3 (Shared region between Meis1 and HOXB13) (FIG. 1A), where key amino acid residues that can create potential binding pocket between Meis1 and HOXB13 interface were identified by THR 296, HIS 297, PRO 298, TYR 299, PRO 300, and SER 301 at Meis1 along with LYS 218m LYS 219, ARG 220, ILE 221, and PHE 222 at HOXB13.

A structure-based drug repurposing platform (FIG. 1B) was applied via docking energy-minimized FDA library against the interacting DNA binding domains (DBDs) between Meis1 and HOXB13 which identified paromomycin, neomycin, rutin, hesperidin, nystatin, kanamycin, amikacin, amphotericin-B, streptomycin, and tobramycin based on their: (1) energy profiles, (2) preferential binding modes, (3) literature survey, and (4) clinical indications and exclusion of undesirable adverse effects. Based on the binding mode, a preliminary structure-binding relationship was elucidated to show that glycosides moieties created a network of hydrogen bonds (acceptors and donors) to enhance the binding affinity of the top candidates towards both of Meis1 and HOXB13 DBDs. TABLE 1 shows interacting energies (AG) for the top 10 clinical candidates identified as targeting S1, S2, and S3 (FIG. 1C).

TABLE 1

| Drug | S1 (Kcal/mol) | S2 (Kcal/mol) | S3 (Kcal/mol) |
|---|---|---|---|
| Paromomycin | −12.8545 | −17.6128 | −10.3948 |
| Neomycin | −15.4412 | −15.7070 | −7.7311 |
| Amikacin | −13.3170 | −14.8337 | −7.6187 |
| Hespiridin | −14.8528 | −14.3192 | −8.6268 |
| Rutin | −12.5358 | −15.4086 | −9.8235 |
| Kanamycin A | −10.0270 | −12.5411 | −7.2004 |
| Nystatin | −14.0836 | −15.2090 | −10.3843 |
| *Tobramycin | −11.0604 | −12.7360 | −7.0629 |
| Amphotericin B | −13.6595 | −14.7172 | −10.8144 |
| Streptomycin | −10.0048 | −10.9770 | −8.7210 |

### Example 2

In another exemplary method, in vitro phenotypic screening for top nine drug candidates for neonatal rat ventricular myocytes (NRVMs) proliferation was performed. Cultured NRVMs were treated with 10 μM of the top candidates were stained with mitotic marker phosphohistone 3 (pH3), troponin (Tnnt), and nuclear marker (DAPI) (FIGS. 1D-1E). Only paromomycin, neomycin, rutin, and hesperidin showed a significant increase in the pH3-positive cardiomyocytes-up to 5-fold compared to control treated NRVMs suggesting their potential to increase the proliferation (FIG. 1E).

### Example 3

In another exemplary method, neomycin, paromomycin, rutin, and hesperidin were screened to evaluate their potential to inhibit Meis1 and HOXB13 transcriptional activity using luciferase assays (FIGS. 1F-11). Two controls were tested due to the solubility of neomycin and paromomycin in PBS, while rutin and hesperidin were soluble in DMSO. Interestingly, neomycin and paromomycin showed significant downregulation for luciferase transcriptional activity for Meis1 and HOXB13 in a dose dependent manner ranging from 10-500 nM; compared to control (PBS) (FIGS. 1F-1G). However, rutin and hesperidin showed poor inhibitory profile for luciferase activity compared to DMSO as control (FIGS. 1H-11). These data suggest that neomycin and paromomycin inhibited Meis1-HOXB13 transcriptional activity. Docked poses for neomycin (Magenta, FIG. 1J) and paromomycin (Green; FIG. 1K) against S1, S2, and S3 sites for Meis1-HOXB13 crystal structure are shown in FIG. 1L.

### Example 4

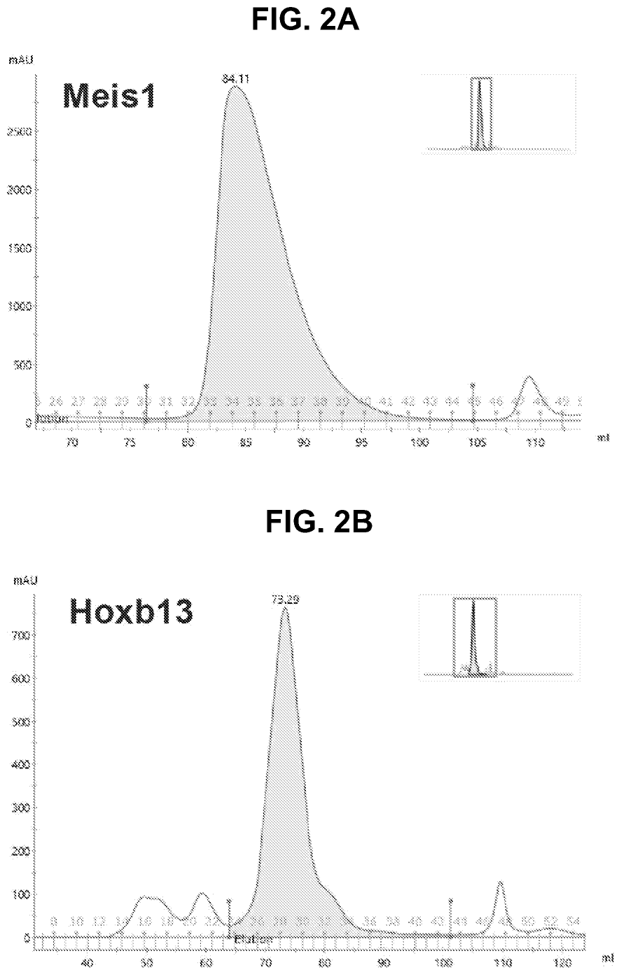

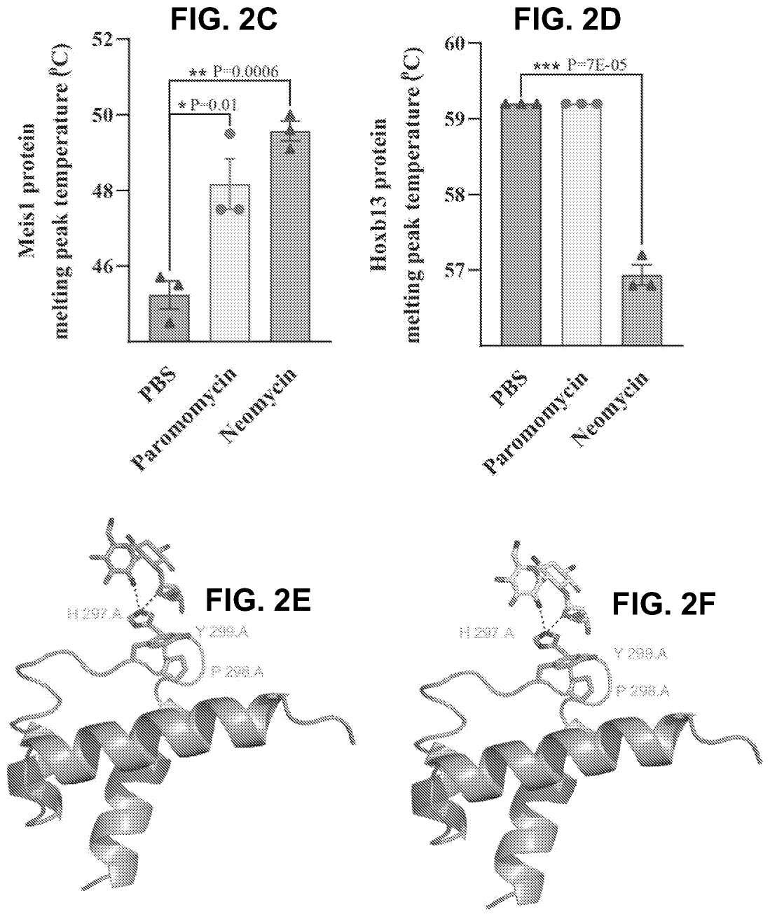

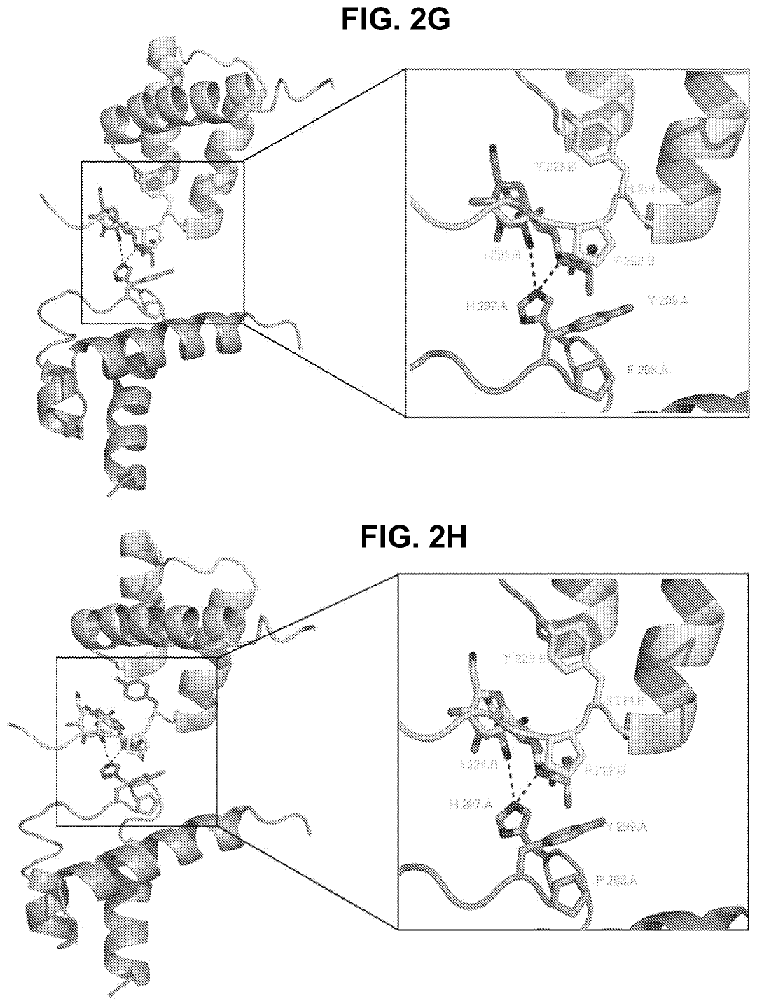

In another exemplary method, thermal shift analysis of aminoglycosides towards Meis1 and HOXB13 was performed. Initially, drug binding domains (DBDs) of Meis1 and HOXB13 were cloned, expressed, and purified to conduct the thermal shift assay (FIGS. 2A-2B). Meis-1 showed a significant change in the melting temperature along with neomycin and paromomycin up to 10° C. in a dose-dependent manner (FIG. 2C). However, HOXB13 showed a noticeable change in the melting temperature along with neomycin solely (FIG. 2D). These data suggest the potential of aminoglycosides to induce conformational change in purified DBDs of Meis1 and HOXB13.

### Example 5

In another exemplary method, the structural basis of Neomycin bound to Meis1 was assessed by X-ray crystallography. In brief, the DBDs of Meis1 were crystallized to elucidate their conformational apo-structure in the absence of DNA. Subsequently, Meis1 crystals were grown in the presence of 10 mM neomycin under conditions that do not support crystal growth for the protein in the absence of neomycin. Density for a fragment of neomycin was identified at a lattice contact near His301 in monomer G. Due to the low level of density beyond the ribose moiety, it was not clear if the bound ligand was neomycin or an impurity such as ribostamycin. Modeled into the density was the D-ribose, D-neosamine and 2-deoxystreptamine moieties of neomycin.

FIGS. 2E and 2G show Meis1 bound to Ribostamycin highlighting the contributing amino acids H297.A, P298.A, and Y299.A whereas FIG. 2F shows HOXB13 bound to Ribostamycin. FIG. 2H shows a close-up image of the binding site with the contributing amino acid residues from HOX13 and Meis1 along with Ribostamycin.

### Example 6

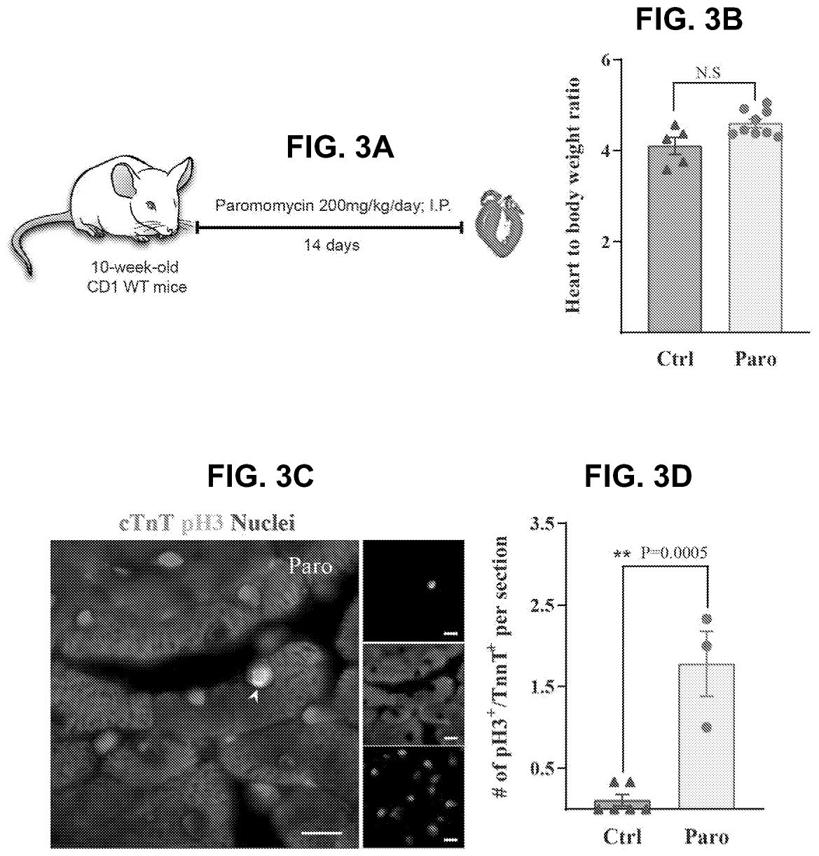

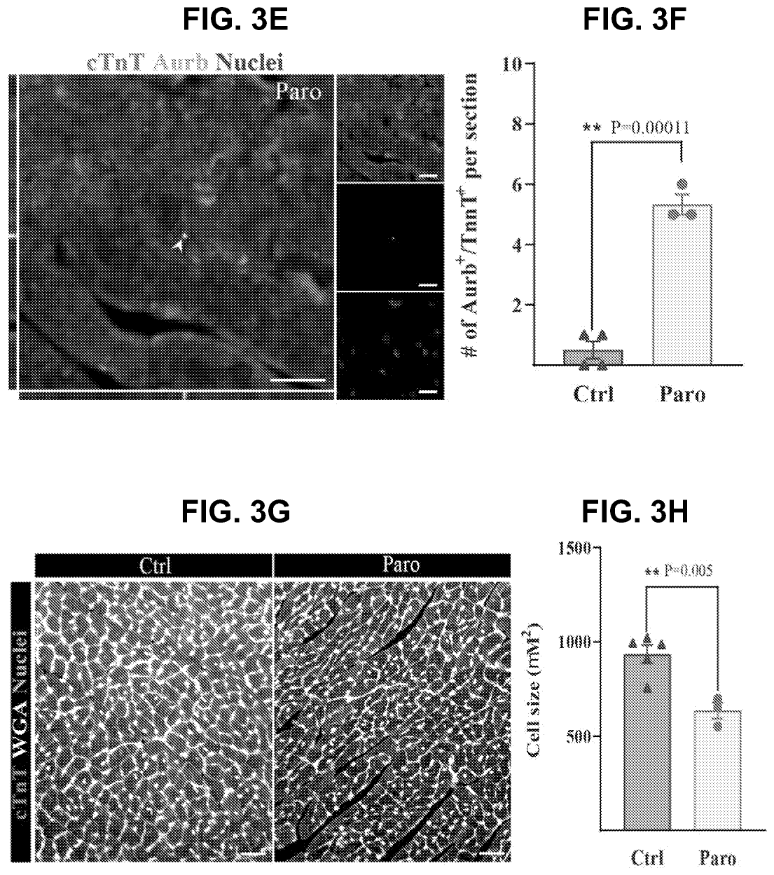

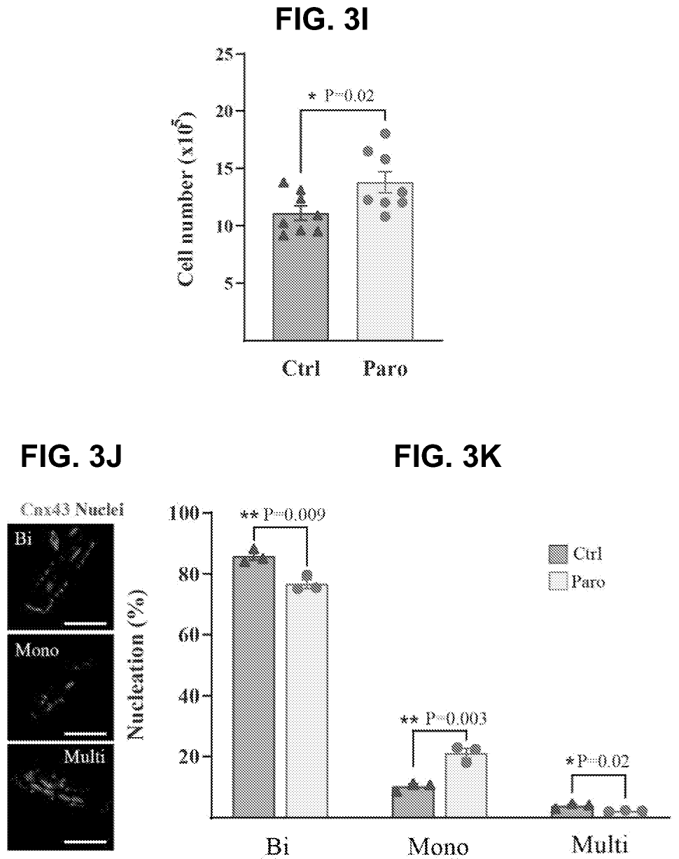

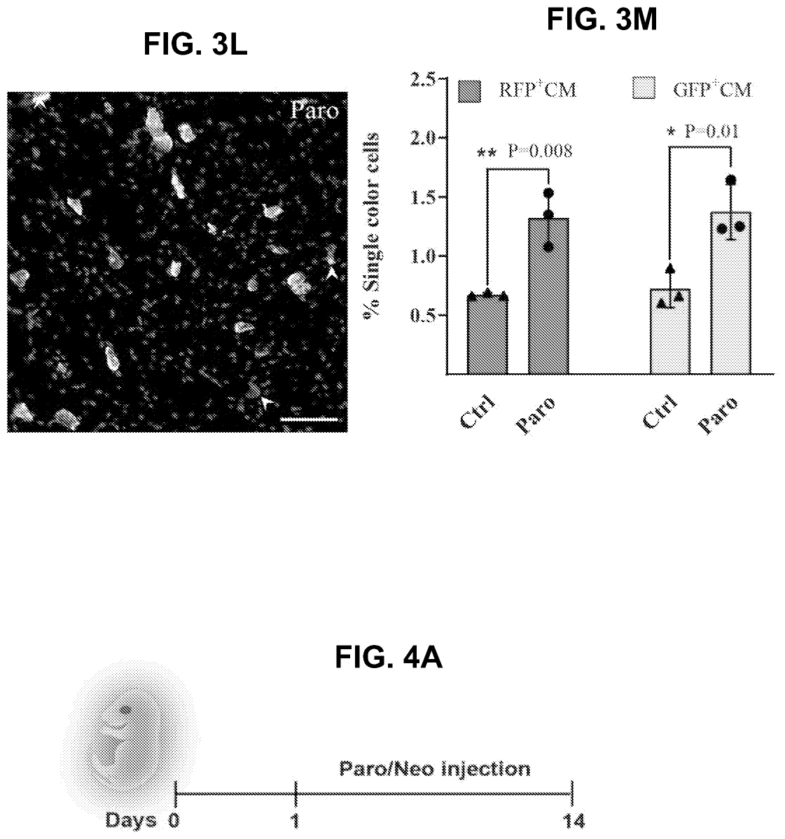

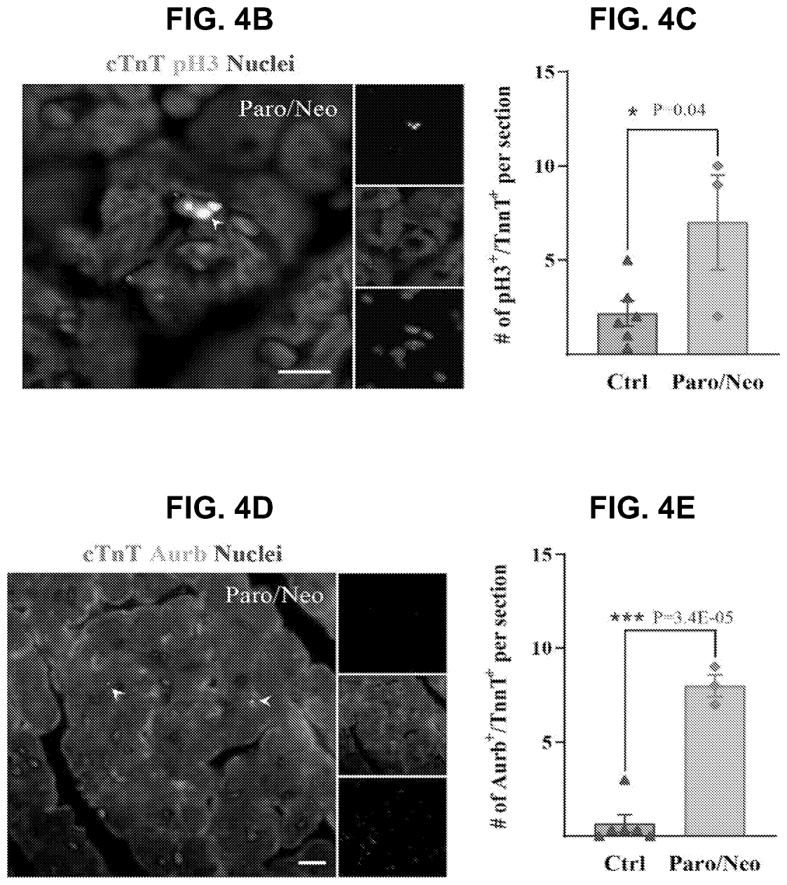

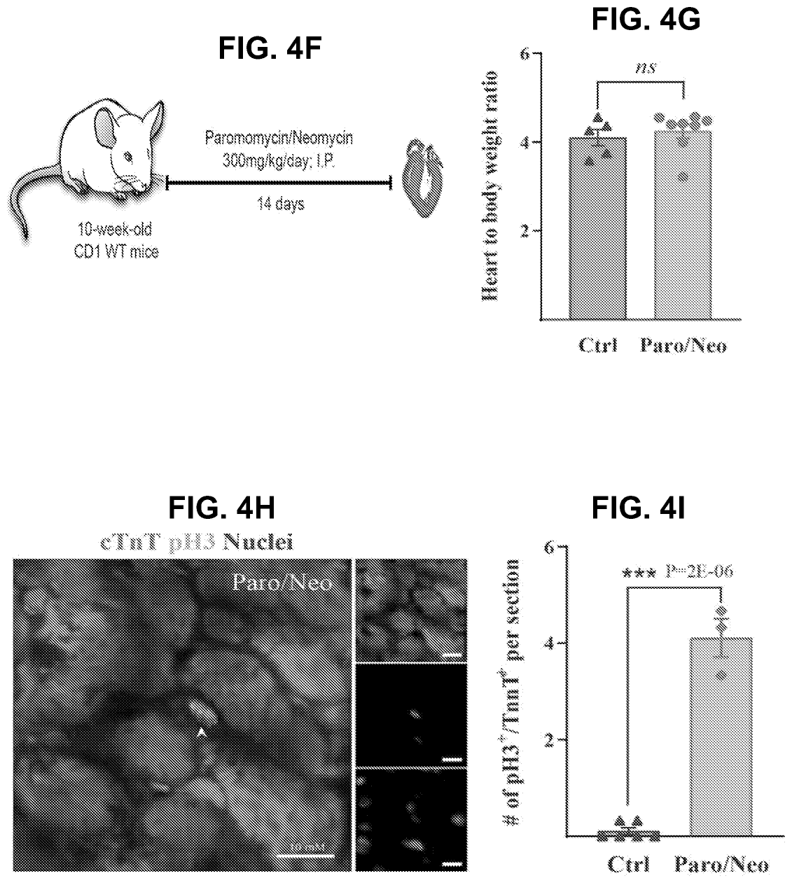

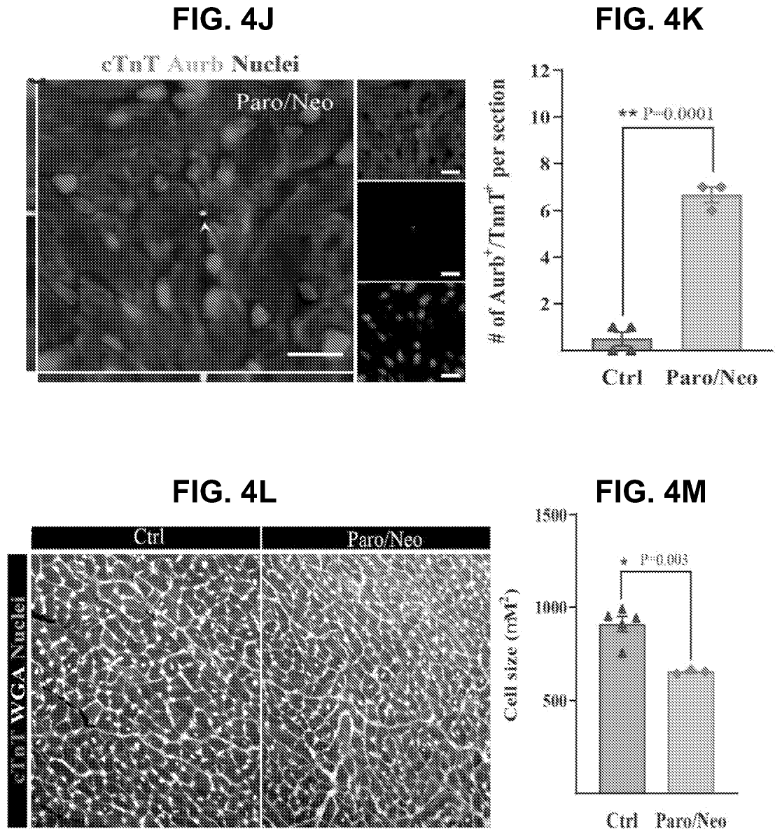

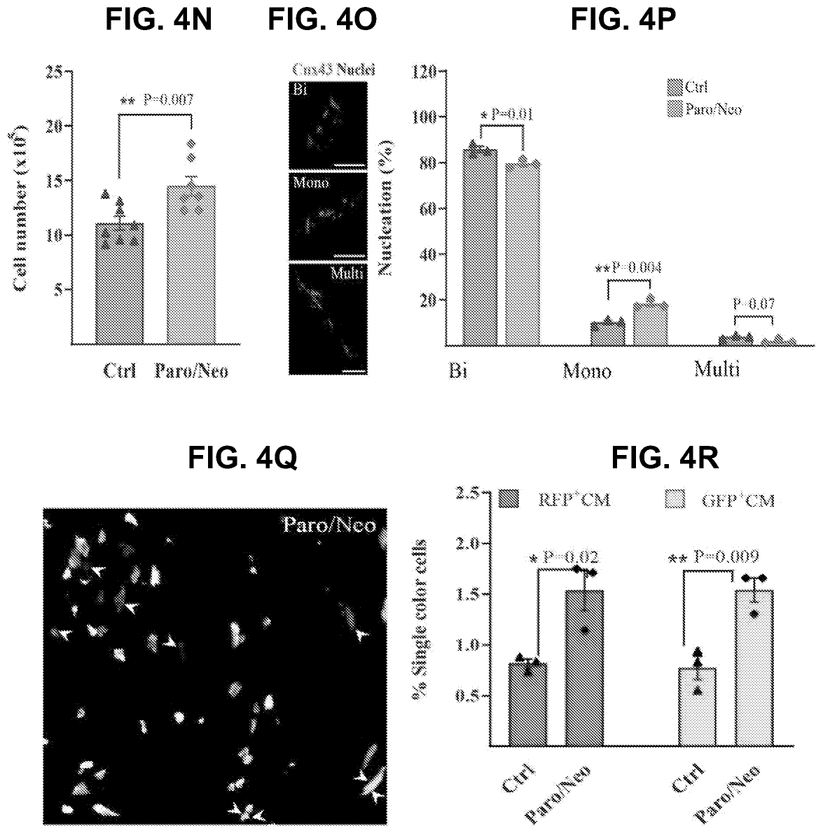

In another exemplary method, cardiomyocyte proliferation in mice was assessed in the presence of paromomycin and paromomycin-neomycin. Based on the data resulting from the in vitro screening coupled and luciferase transcription activity assays detailed in the exemplary methods herein, paromomycin was initially selected to study its capacity to prolong cardiomyocytes proliferation in adult mice with no injury stimuli. The effect of paromomycin (Paro) (200 mg/kg/day, i.p.) and paromomycin-neomycin (Paro/Neo) combo (300 mg/kg/day, i.p.) was assessed after 2 weeks of injection on cell proliferation indices, cytokinesis, cardiomyocyte cell size, isolated cardiac cell count, and nucleation on the isolated cardiomyocytes in the adult heart (FIG. 3A, FIG. 4F). Overall, no significant difference was found in heart weight (HW)/body weight (BW) ratio in Paro and Paro/Neo combo-treated mice, compared to control-treated mice (FIG. 3B, FIG. 4G). Paro and Paro/Neo combo showed significant increase in pH3⁺, compared to control-treated mice to suggest their potential to increase proliferation (FIGS. 3C-3D, FIGS. 4H-4I). Also, Paro and Paro/Neo combo showed significant increase in cardiomyocytes undergoing cytokinesis via Aurkb⁺ staining (FIGS. 3E-3F, FIGS. 4J-4K). In addition, Paro and Paro/Neo combo showed significant reduction in cardiomyocytes cell size via wheat germ agglutinin (WGA) staining (FIGS. 3G-3H, FIGS. 4L-4M), suggesting the formation of newly cardiomyocytes. Furthermore, Par and Par/Neo combo-treated hearts had a significantly higher number of cardiomyocytes following dissociation of the heart by collagenase digestion, compared to control-treated hearts (FIG. 3I, FIG. 4N). To validate the aminoglycosides proliferative capacity to generate new cardiomyocytes, a lineage-tracing system was used for assessment of Paro and Paro/Neo combo using Mosaic Analysis with Double Markers (MADM) mice to show significant increased RFP⁺ and GFP⁺ single-labelled daughter cardiomyocytes, compared to control-treated mice (FIGS. 3J-3K, FIGS. 4Q-4R).

Paromomycin-neomycin was administered to neonatal CD-1 mice at 300 mg/kg, i.p from p1 till p14. Hearts were collected for histological analysis (FIG. 4A). Anti-pH3 and anti-cardiac troponin T (anti-cTnT) co-immunostaining showed a significant increase in the cardiomyocyte mitosis marker for paromomycin-treated neonates, compared with controls (FIGS. 4B-4C). AurkB and anti-cTnT co-immunostaining showed a significant increase in the cardiomyocyte cytokinesis marker for paromomycin-treated neonates, compared with controls (FIGS. 4D-4E).

### Example 7

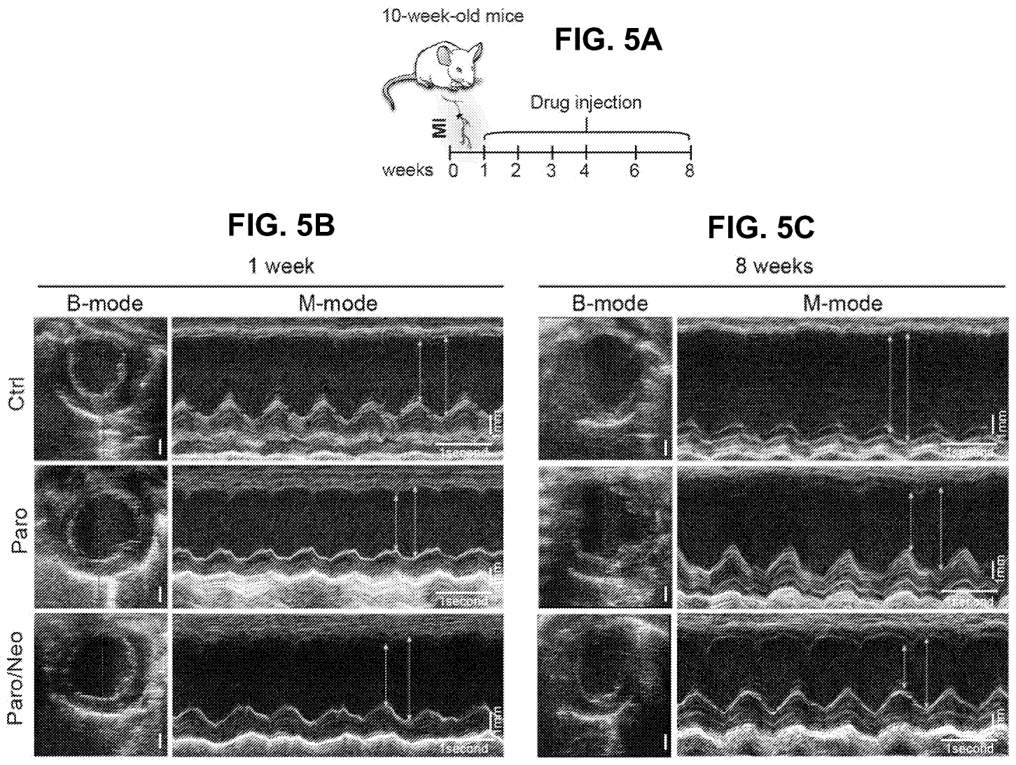

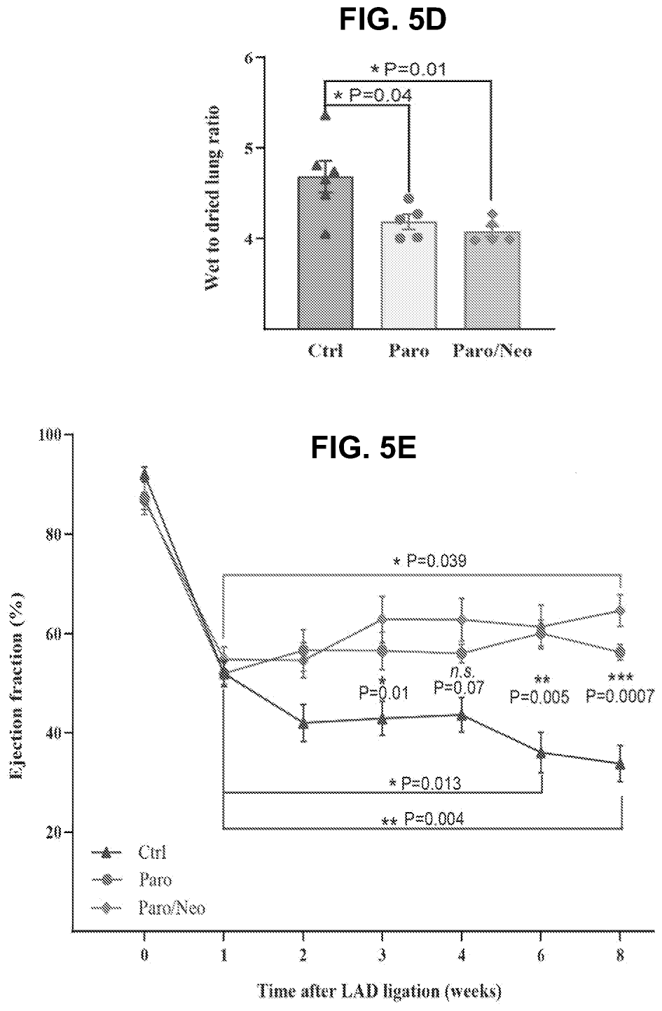

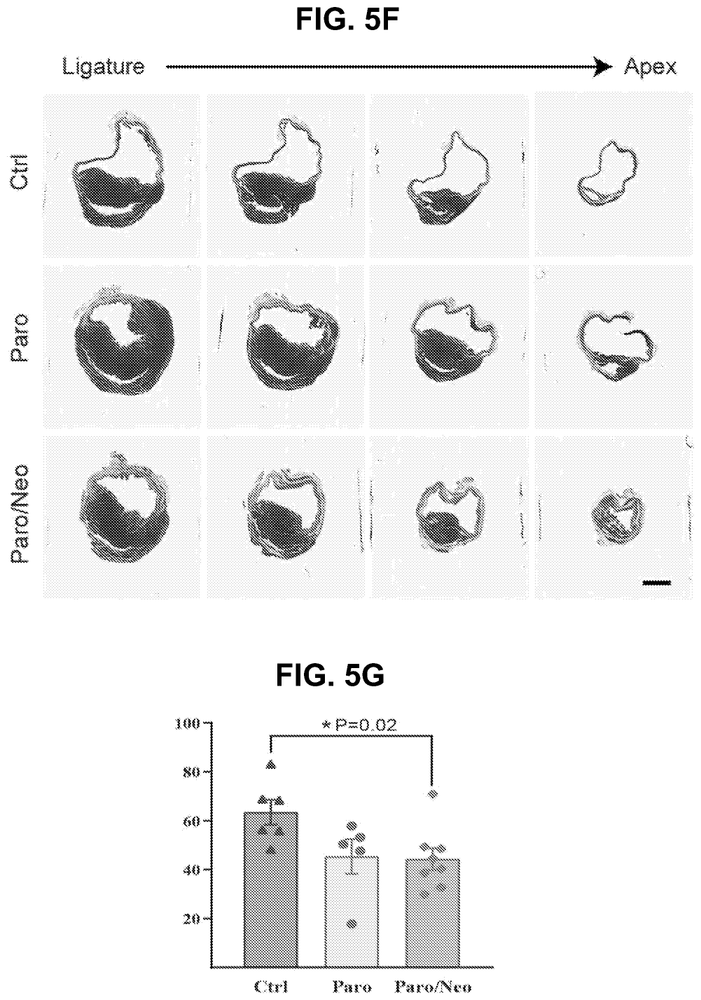

In another exemplary method, the ability of the aminoglycosides on cardiac repair and heart regeneration was assessed in the mouse adult heart after following myocardial infarction (MI). MI was induced in adult mice by permanent ligation of the proximal left anterior descending coronary artery. This was followed by examining the expression of cell cycle inhibitors two weeks after MI (one week after drug(s) injection) by western blot analysis. This revealed a post-MI decrease in p21 and p15/16 proteins in the paromomycin and paromomycin-neomycin combo therapeutic regimen. Meanwhile, MI was induced to monitor the pharmacodynamics of the administered drug(s) to improve cardiac function and promote heart regeneration for 8-weeks (FIG. 5A). One week after injury, paromomycin and paromomycin-neomycin combo (200 mg/kg/day, i.p.) was administered while assessment of left ventricular ejection fraction (LVEF) by echocardiography was performed 1-week post-surgery, 1 week after drug(s) administration and every 2 weeks thereafter until 8 weeks post-surgery.

By 8 weeks post-surgery, paromomycin prevented cardiac remodelling post-MI to maintain the LVEF, compared to the control-treated mice (FIGS. 5B-5C). Paro/Neo combo-treated mice showed a significant improvement in LVEF with reduced left ventricular dilatation (FIG. 5D). Then, the infarcted hearts treated with control (PBS), Paro, and Paro/Neo combo, where Paro-treated hearts were subjected to histological analysis. The data showed a trending decline in the fibrotic scar, compared to control-treated heats (FIG. 5E).

Meanwhile, a significant decrease in LV dilatation and remodeling of the Paro/Neo combo treated hearts were determined and quantified, compared to the control-treated hearts, seen by Trichrome staining at 8 weeks post-MI with significant reduction in the fibrotic scar size (FIGS. 5F-5G). These data suggest that Par/Neo combo restored the cardiac function and structure to prevent cardiac remodelling post-MI.

### Example 8

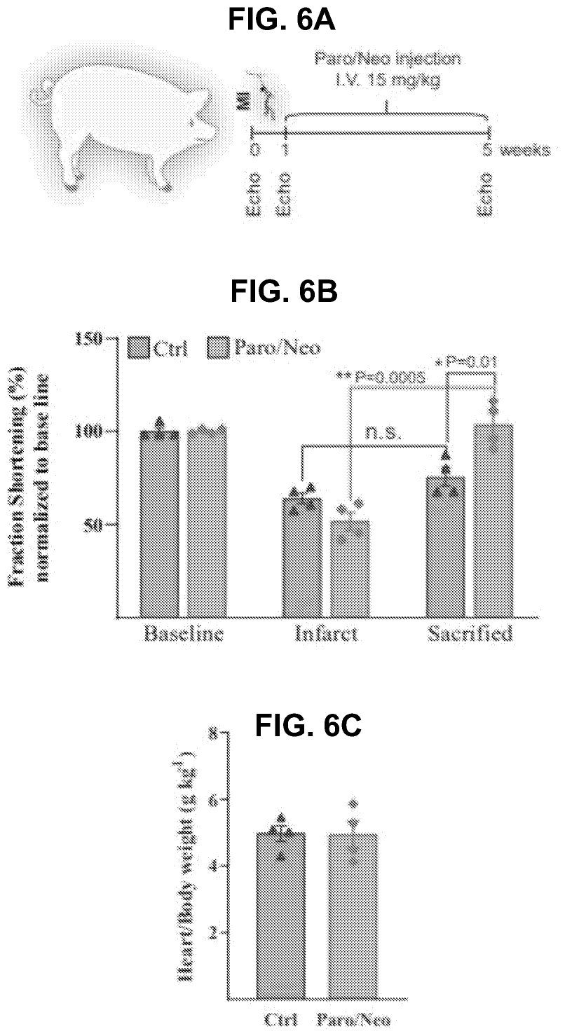

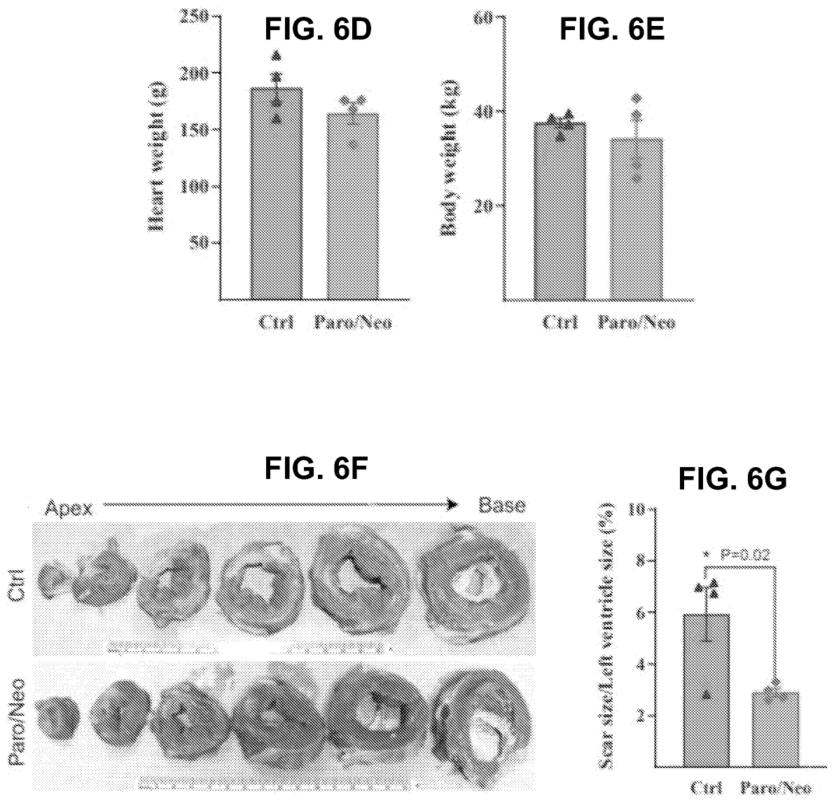

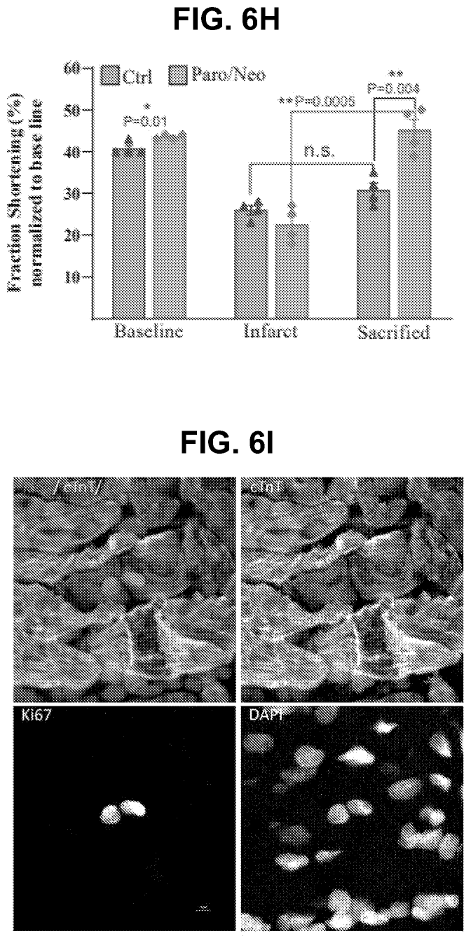

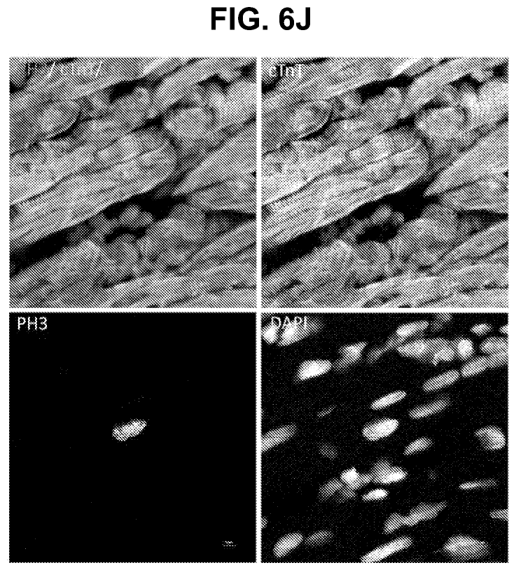

In another exemplary method, the efficiency of aminoglycosides on cardiac repair and heart regeneration was assessed in the pig adult heart after following myocardial infarction (MI). In brief, MI was induced in adult pigs, followed by examining (LVEF) by echocardiography 1-week post-surgery, 1 week after drug(s) (Paromomycin-neomycin, 15-16 mg/kg, i.v.) administration and every 2 weeks thereafter until 4 weeks post-surgery (FIG. 6A). By 4 weeks post-surgery, paromomycin-neomycin prevented cardiac remodelling post-MI to maintain the LVEF, compared to the control-treated pigs. Paro/Neo combo-treated pigs showed a significant improvement in LVEF with reduced left ventricular dilatation (FIGS. 6B-6E). Then, infarcted hearts were collected from pigs treated with control (PBS) and Paro/Neo combo, where Paro-Neo treated hearts showed a significant decline in the fibrotic scar, compared to control-treated heats (FIGS. 6F-6H). Also observed in these heart tissues was a significant increase in the cardiomyocytes undergoing proliferation (Ki67⁺ and pH3⁺) and cytokinesis (Aurkb⁺/Tnnt) (FIGS. 6I-6J). These data suggested that Par/Neo combo restored the cardiac function and structure to prevent cardiac remodelling post-MI in large animals.

### Methods Used in Examples 1-8

The following exemplary methods were performed as described in Examples 1-8 herein.

FDA-approved small molecules preparation. The US FDA-approved drug database was downloaded (https://go.drugbank.com/), and three-dimensional structures were energy minimized using MMFF94 force field (63). X-ray crystal structure preparation. Crystal structure of Meis1-HOXB13 has been resolved in the Protein data bank (PDB code: 5EGO).

Structure-based in silico screening and scoring. The top ten selected energy minimized approved small molecules, based on literature survey and clinical indications underwent docking simulations using MOE along with S1, S2, and S3 (PDB ID; 5EGO). The top selected energy minimized compounds underwent protonation state to add the missing hydrogens for proper ionization states (64, 65). MOE Dock application was used to find the favourable binding conformations/poses for the studied candidates. The scoring assessment was conducted by validating the docked poses using the London dG scoring method to estimate the energy profile showing the potential hydrophobic and hydrogen bond interactions. Three-dimensional visualization was generated using Pymol (66), while the 2D generation was done using MOE tools.

Neonatal Rat Ventricular myocytes (NRVM) isolation and cell culture. Cardiomyocytes were isolated from the left ventricle of 1-2-day-old Sprague-Dawley rats following the protocol from an isolation kit (Cellutron Life Technologies, cat #nc-6031). Myocytes were then plated on cover slips coated with laminin (Life tech #23017-015) at a density of 1250 cells/mm in DMEM: M199 (3:1) containing 10% horse serum, 5% FBS, and 1% Penicillin/Streptomycin. 100 μmol/L 5′-bromo-2′-deoxyuridine is included to inhibit growth of fibroblast. 24 hrs after cell attachment, change culture medium.

Animals. Mice were housed in a 12:12 h light: dark cycle in a temperature-controlled room with free access to water and food. Littermate controls were used whenever possible. Both male and female mice were used on age-matched and gender-matched mice. No statistical methods were used to predetermine sample size. All surgeries and echocardiographic studies were carried out blinded to the genotype of the mice during the experiments and outcome assessments. CD1 mice (Charles River Laboratories) were used for drug-tested studies.

Drug administration. Tamoxifen (Sigma) was prepared by dissolving in sesame oil (Sigma) to a concentration of 10 mg ml⁻¹. For the MADM clonal analysis, 14 consecutive injections of tamoxifen were administered, and mice were euthanized 2 weeks after the last injection. For injection of Paromomycin and Neomycin, mice were administered twice daily. Mice were euthanized at the same time of day to limit circadian variability.

Murine model of MI. Induction of adult anterior wall MI was performed as similar to the methods described in Nguyen et al., Nature (2020) 582, 271-276, the disclosure of which is incorporated herein in its entirety. In brief, three-month-old mice were subjected to MI by ligation of the proximal aspect of the LAD coronary artery. Mice were anaesthetized in an airtight chamber using 4% isoflurane, endotracheally intubated, and ventilated using a volume-control ventilator with 100% O₂, supplemented with 2% vaporized isoflurane (Harvard Apparatus). Following lateral thoracotomy and pericardiectomy, the LAD coronary artery was identified along the anterior wall of the LV. Prolene sutures (6-0 non-absorbable) was used to ligate the LAD. Proper occlusion of the LAD artery was noted by three criteria: immediate blanching of the LV anterior wall myocardium below the ligature, echocardiographic assessment 7 days post-surgery showing an ejection fraction in the range of 45-65%, and histological analysis. Vicryl sutures (6-0 absorbable) were used to close the thoracic cavity. Drugs administrated one week after MI. Mice were subjected to echocardiography at serial time points and then euthanized at 3 months after ligation. Hearts were collected and processed for histology and immunohistochemistry. Fibrotic scar size was measured using MIQuant similar to the method described in Nascimento et al., PLOS One (2011) 6, e25045, the disclosure of which is incorporated herein in its entirety.

Transthoracic echocardiography. Assessment of in vivo heart function on conscious, non-sedated mice was performed using a Vevo2100 micro-ultrasound system, MS400C probe (VisualSonics) at baseline, 1 week after injury, 1 week after drug administration, and 4, 8, 12 and 16 weeks after occlusion. Echocardiographic M-mode images were obtained from a parasternal short-axis view at the level of the papillary muscles. Left ventricular internal diameters at end diastole (LVIDd) and end systole (LVIDs) were measured from M-mode recordings. Six representative contraction cycles were selected for analysis, and average indexes (LVIDd, LVIDs and fractional shortening) were calculated for each mouse. All echocardiography measurements were performed in a blinded manner.

Histology. Hearts were collected and fixed in 4% paraformaldehyde in PBS overnight at 4° C. and then processed for either paraffin or cryo embedding. H&E and Masson's trichrome staining were performed according to standard procedures on paraffin sections.

Immunofluorescence staining. Heart cryosections were equilibrated with antigen retrieval buffer with 1 mM EDTA or epitope retrieval buffer (IHC World). Samples were permeabilized and blocked with 0.3% Triton X-100 and 10% serum from the host animal of secondary antibodies in PBS for 20 minutes at room temperature. Then, samples were incubated overnight at 4° C. with the primary antibodies. After three washes with PBS, samples were incubated for 1 hour at room temperature with corresponding fluorescence secondary antibodies conjugated to Alexa Fluor 488 or 555 (Invitrogen) at 1:400. The slides were mounted in Vectashield Antifade Mounting Medium (Vector Laboratories). Slides were viewed under Nikon fluorescence or Zeiss LSM 510 confocal microscopes. Primary antibodies used: phospho histone H3 Ser10 (EMD Millipore, 06-570; 1:100), aurora B kinase (Sigma, A5102; 1:25) to analyse cell cycle re-entry; troponin T, cardiac isoform Ab-1, clone 13-11 (Thermo Scientific, MS-295-P1; 1:200), sarcomeric α-actinin (Abcam, ab68167; 1:200) to identify cardiomyocytes. DAPI or Hoechst was used for nuclear staining. Images were obtained using a Nikon Eclipse Ni or Nikon A1 laser scanning confocal microscopes.

WGA staining and cardiomyocyte size quantification. WGA staining and quantification was performed as previously described. In brief, the slides were incubated with WGA conjugated to Alexa Fluor 488 (50 mg ml⁻¹, Life Technologies) for 1 hour at room temperature following washing with PBS. To quantify the cross-sectional cell size, three to five independent hearts per group with three different views and positions, each from left and right ventricles, and septum were captured at 40× magnification. ImageJ was used to quantify the size of cardiomyocytes that were round and contained a nucleus. At least 500 cells per sample were quantified.

Cardiomyocyte isolation. Adult hearts were freshly collected and fixed in 4% paraformaldehyde at 4° C. overnight. The hearts were minced to smaller pieces and subsequently incubated with collagenase D (2.4 mg ml⁻¹, Roche) and B (1.8 mg ml⁻¹, Roche) for 12 h at 37° C. using an end-over-end shaker. The supernatants were collected via 160-μm nylon mesh filter, and the procedure was repeated until no more cardiomyocytes were dissociated from the tissue. The isolated cardiomyocytes were co-stained with connexin 43 (IHCWorld, IW-PA1026; 1:100) and DAPI (Sigma, D9542; 1:10,000) for further quantification. For nucleation counts, at least 300 cardiomyocytes per sample were quantified.

Western blotting. Ventricles were collected and lysed in RIPA buffer with the addition of complete protease inhibitor cocktail (Roche). Protein concentration was quantified using Pierce BCA protein assay kit (Pierce Biotechnology), with three biological replicates. After separation via SDS-PAGE, proteins were transferred to nitrocellulose membranes (Bio-Rad), blocked in 5% skim milk/TBS and incubated with appropriate primary antibodies: p21 Waf1/Cip1 (12D1) (Cell Signaling, 2947; 1:1,000), p15/16 (Santa Cruz, sc-377412; 1:1,000), Gapdh (EMD Milipore, AB2302; 1:5,000), α-tubulin (Milipore, 04-1117; 1:2,000). Horseradish peroxidase-conjugated peroxidase anti-mouse, anti-rabbit, anti-chicken or anti-goat antibodies (Jackson ImmunoResearch, 115-035-166, 111-035-144, 703-035-155, 705-035-147; 1:25,000-1:50,000) were used as secondary antibodies. The membranes were explored using Licor Odyssey Fc system and quantified by Image Studio Lite v.5.2 software.

Protein Expression and Purification. mMeis1 and mHoxb13 were subcloned into pETDuet vector with noncleavable N-terminal 6×His tag and transformed into Rosetta (DE3) pLysS cells (Novagen). Target protein were expressed in cultures grown in autoinduction media at 18° C. overnight. The culture was harvested and sonicated in lysis buffer (20 mM Tris [pH=8.0], 1 M NaCl, 0.5 mM DTT, and supplemented with protease inhibitors). The lysate was centrifuged, the supernatant was loaded onto a Ni-NTA affinity column (Qiagen), and the beads were washed with wash buffer (20 mM Tris [pH=8.0], 1 M NaCl, 0.5 mM DTT, and 20 mM Imidazole [pH=8.0]) and eluted with elution buffer (20 mM Tris [pH=8.0], 150 mM NaCl, 0.5 mM DTT, and 250 mM Imidazole [pH=8.0]). The eluate was concentrated and purified by gel filtration chromatography. The peak fractions were collected and concentrated to about . . . 0 mg/mL for crystallization screening.

Crystallization and structure determination. For neomycin bound Meis1, recombinant Meis1 in 5 mM Tris-HCl pH 8, 30 mM NaCl was concentrated to 10 mg/mL. The crystals were grown at 20° C. by the hanging drop vapor diffusion method using a 1:1 ratio of protein: reservoir solution containing 18% PEG3350, 0.2 M LiCl, and 10 mM neomycin and were flash frozen in 20% PEG3350, 0.2 M LiCl, 30 mM NaCl and 30% ethylene glycol. Meis2 crystals exhibited the symmetry of space group P3₂12 with cell dimensions of a=77.76 Å, c=213.30 Å, contained 8 monomers (as 4 dimers) per asymmetric unit, and diffracted to a minimum Bragg spacing (dₘᵢₙ) of 2.45 Å when exposed to synchrotron radiation. Diffraction data were collected at 100 K at the Advanced Photon Source beamline 19-ID. Data were indexed, integrated, and scaled using the HKL-3000 program package (Minor et al., Acta Crystallographica. Section D, Biological Crystallography (2006) 62:859-866).

Phase determination, modeling and structure refinement. Phases for neomycin bound homeobox domain of Meis1 were obtained via molecular replacement in the program Phaser (McCoy et al., Journal of Applied Crystallography (2007) 40:658-674) using the previously deposited Meis2 dimer coordinates as a search model (PDB ID 3K2A, DOI: 10.2210/pdb3K2A/pdb). Model rebuilding was performed in the program Coot (Emsley et al., Acta Crystallographica. Section D, Biological Crystallography (2010) 66:486-501. doi: 10.1107/S0907444910007493). Positional and isotropic atomic displacement parameter (ADP) as well as TLS ADP refinement was performed to a resolution of 2.45 Å using the program Phenix (Afonine et al., Acta Crystallographica. Section D, Biological Crystallography (2010) 66:1153-1163) with a random 8% of all data set aside for an R_{free} calculation. The current model contained 4 Meis2 dimers; included were residues 280-338, one neomycin fragment and 61 water molecules. The R_{work} is 0.216 and the R_{free} is 0.256. A Ramachandran plot generated with Molprobity (Chen et al., Acta Crystallographica. Section D, Biological Crystallography (2010) 66:12-21.) indicated that 97.9% of all protein residues were in the most favored regions and none in disallowed regions.

Statistics and Reproducibility. Differences between groups were examined for statistical significance using unpaired two-sided Student's t-test. All bar graphs represent mean±s.e.m. Statistical significance was assessed as *P<0.05, ** P<0.01 and *** P<0.001. P values are shown in graphs.

1. A method of inducing regeneration of cardiomyocytes in a heart tissue comprising contacting the heart tissue with a pharmaceutical composition comprising one or more aminoglycosides and at least one pharmaceutically acceptable carrier.

2. The method according to claim 1, wherein the one or more aminoglycosides comprises amikacin, gentamicin, tobramycin, streptomycin, neomycin, kanamycin, netilmicin, paromomycin, spectinomycin, or any combination thereof.

3. The method according to claim 2 comprising two aminoglycosides.

4. The method according to claim 3, wherein the two aminoglycosides comprise paromomycin and neomycin.

5. (canceled)

6. The method according to claim 1, wherein contacting the heart tissue with the pharmaceutical composition increases cardiomyocyte proliferation by about 1-fold to about 10-fold compared to a heart tissue not contacted with the one or more aminoglycosides.

7. A method of improving cardiac repair of heart tissue in a subject in need thereof, the method comprising administering to the subject an effective amount of a pharmaceutical composition comprising one or more aminoglycosides and at least one pharmaceutically acceptable carrier wherein the subject has or is suspected of having a myocardial injury.

8. The method according to claim 7, wherein the one or more aminoglycosides comprises amikacin, gentamicin, tobramycin, streptomycin, neomycin, kanamycin, netilmicin, paromomycin, spectinomycin, or any combination thereof.

9. The method according to claim 8 comprising two aminoglycosides.

10. The method according to claim 9, wherein the two aminoglycosides comprise paromomycin and neomycin.

11. (canceled)

12. The method according to claim 7, wherein the pharmaceutical composition is administered to the subject by a parenteral route.

13. The method according to claim 7, wherein the myocardial injury is selected from the group consisting of arterial disease, atheroma, atherosclerosis, arteriosclerosis, coronary artery disease, arrhythmia, angina pectoris, congestive heart disease, ischemic cardiomyopathy, myocardial infarction, stroke, transient ischemic attack, aortic aneurysm, cardiopericarditis, bacterial infection, viral infection, inflammation, valvular insufficiency, and vascular clotting defects.

14. The method according to claim 7, wherein administration of an effective amount of the pharmaceutical composition increases life expectancy of the subject compared to an untreated subject with identical disease condition and predicted outcome.

15. The method according to claim 7, wherein administration of an effective amount of the pharmaceutical composition improves heart function of the subject compared to an untreated subject with identical disease condition and predicted outcome.

16. The method according claim 7, wherein administration of an effective amount of the pharmaceutical composition reduces cardiac fibrosis in the subject compared to an untreated subject with identical disease condition and predicted outcome.

17. The method according to claim 7, wherein administration of an effective amount of the pharmaceutical composition reverses cardiac hypertrophy in the subject compared to an untreated subject with identical disease condition and predicted outcome.

18. The method according to claim 7, wherein the subject is a human patient having or suspected of having a myocardial injury.

19. (canceled)

20. The method according to claim 7, wherein the pharmaceutical composition is administered to the subject immediately after myocardial injury.

21. The method according to claim 7, wherein the pharmaceutical composition is administered to the subject immediately up to about one week after myocardial injury.

22. The method according to claim 7, wherein the pharmaceutical composition is administered to the subject by a schedule ranging from about three times per day to about every other week.

23. The method according to claim 7, wherein the pharmaceutical composition is administered to the subject at a dose ranging from about 50 mg/kg/day to about 500 mg/kg/day.

24. (canceled)

25. (canceled)

26. (canceled)

27. (canceled)

28. (canceled)

29. (canceled)

30. (canceled)
